SEQUENCE LISTING (89 sequences)

SEQ ID NO: 1 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 2 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 3 - protein, 349 aa, Streptococcus pyogenes
MKTIAYPNKPHSLSAGLLTAIAIFSLASSNITYADDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 4 - protein, 315 aa, Streptococcus pyogenes
DDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 5 - protein, 313 aa, Streptococcus pyogenes
DDYQRNATEAYAKEVPHQITSVWTKGVTPLTPEQFRYNNEDVFHAPYVANQGWYDITKAFDGKDNLLCGAATAGNMLHWWFDQNKDQIKRYLEEHPEKQKINFNGDNMFDVKKAIDTKNHQLDSKLFNYFKEKAFPGLSARRIGVFPDHVIDMFINGYRLSLTNHGPTPVKEGSKDPRGGIFDAVFTRGNQSKLLTSRHDFKNKNLNDISTIIKQELTKGKALGLSHTYANVSINHVINLWGADFNAEGNLEAIYVTDSDSNASIGMKKYFVGVNAHGHVAISAKKIEGENIGAQVLGLFTLSTGQDSWQKLS

SEQ ID NO: 6 - protein, 315 aa, Streptococcus pyogenes
DDYQRNATEAYAKEVPHQITSVWTKGVTPLTPEQFRYNNEDVIHAPYLANQGWYDITKAFDGKDNLLCGAATAGNMLHWWFDQNKTEIEAYLSKHPEKQKIIFRNQELFDLKEAIRTKDSQTNSQLFEYFRDKAFPYLSARQLGVMPDLVLDMFINGYYLNVFKTQSTDVKRPYQDKDKRGGIFDAVFTRGNQTTLLTARHDLKNKGLNDISTIIKEELTKGRALALSHTYANVSISHVINLWGADFNAEGNLEAIYVTDSDANASIGMKKYFVGINKHGHVAISAKKIEGENIGAQVLGLFTLSSGKDIWQKLN

SEQ ID NO: 7 - protein, 315 aa, Streptococcus pyogenes
DDYQRNATEAYAKEVPHQITSVWTKGVTPLTPEQFRYNNEDVIHAPYLAHQGWYDITKTFNGKDNLLCGAATAGNMLHWWFDQNKTEIEAYLSKHPEKQKIIFNNEELFDLKAAIDTKDSQTNSQLFNYFKEKAFPNLSTRQLGVMPDLVLDMFINGYYLNVFKTQSTDVNRPYQDKDKRGGIFDAVFTRGNQTTLLTARHDFKEKGLKDISTIIKQELTEGRALALSHTYANVSISHVINLWGADFDAEGNLKAIYVTDSDANASIGMKKYFVGINAHGKVAISAKKIEGENIGAQVLGLFTLSSGKDIWQQLS

SEQ ID NO: 8 - protein, 315 aa, Streptococcus pyogenes
DSFSANQEIRYSEVTPYHVTSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 9 - protein, 295 aa, Streptococcus pyogenes
SVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 10 - protein, 314 aa, Streptococcus pyogenes
DDYQRNATEAYAKEVPHQITSVWTKGVTPLTPEQFTQGEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 11 - protein, 312 aa, Streptococcus pyogenes
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 12 - protein, 313 aa, Streptococcus pyogenes
DDYQRNATEAYAKEVPHQITSVWTKGVTPPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 13 - protein, 312 aa, Streptococcus pyogenes
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIINNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 14 - protein, 312 aa, Streptococcus pyogenes
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 15 - protein, 312 aa, Streptococcus pyogenes
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIIRNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 16 - protein, 312 aa, Streptococcus pyogenes
DDYQRNATEAYAKEVPHQITSVWTKGVTPPEQFTQGEDVIHAPYLANQGWYDITKAFDGKDNLLCGAATAGNMLHWWFDQNKTEIEAYLSKHPEKQKIIFRNQELFDLKEAIRTKDSQTNSQLFEYFRDKAFPYLSARQLGVMPDLVLDMFINGYYLNVFKTQSTDVKRPYQDKDKRGGIFDAVFTRGNQTTLLTARHDLKNKGLNDISTIIKEELTKGRALALSHTYANVSISHVINLWGADFNAEGNLEAIYVTDSDANASIGMKKYFVGINKHGHVAISAKKIEGENIGAQVLGLFTLSSGKDIWQKLN

SEQ ID NO: 17 - protein, 292 aa, Streptococcus pyogenes
SVWTKGVTPPEQFTQGEDVIHAPYLAHQGWYDITKAFDGKDNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 18 - protein, 312 aa, Streptococcus pyogenes
DDYQRNATEAYAKEVPHQITSVWTKGVTPPEQFTQGEDVIHAPYLAHQGWYDITKAFDGADNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 19 - protein, 292 aa, Streptococcus pyogenes
SVWTKGVTPPEQFTQGEDVIHAPYLAHQGWYDITKAFDGADNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 20 - protein, 312 aa, Streptococcus pyogenes
DDYQRNATEAYAKEVPHQITSVWTKGVTPPEQFTQGEDVIHAPYLAHQGWYDITKAFDGKDNLLCGAATAGNMLHWWFDQNKTEIEAYLSKHPEKQKIIFRNQELFDLKAAIDTKDSQTNSQLFNYFRDKAFPNLSARQLGVMPDLVLDMFINGYYLNVFKTQSTDVNRPYQDKDKRGGIFDAVFTRGNQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLS

SEQ ID NO: 21 - protein, 312 aa, Streptococcus pyogenes
DDYQRNATEAYAKEVPHQITSVWTKGVTPLTPEQFTQGEDVFHAPYVANQGWYDITKAFDGKDNLLCGAATAGNMLHWWFDQNKDQIKRYLEEHPEKQKINFNGENMFDVKKAIDTKNHQLDSKLFNYFKEKAFPYLSAKHLGVFPDHVIDMFINGYRLSLTNHGPTPVKEGSKDPRGGIFDAVFTRGNQSKLLTSRHDFKNKNLNDISTIIKQELTKGKALGLSHTYANVRINHVINLWGADFNAEGNLEAIYVTDSDSNASIGMKKYFVGVNAHGHVAISAKKIEGENIGAQVLGLFTLSTGQDSWQKLS

SEQ ID NO: 22 - protein, 312 aa, Streptococcus pyogenes
DDYQRNATEAYAKEVPHQITSVWTKGVTPLTPEQFTQGEDVFHAPYVANQGWYDITKAFDGKDNLLCGAATAGNMLHWWFDQNKDQIKRYLEEHPEKQKINFRGENMFDVKEAIRTKNHQLDSKLFEYFKEKAFPYLSAKHLGVFPDHVIDMFINGYRLSLTNHGPTPVKKGSKDPRGGIFDAVFTRGNQSKLLTSRHDFKNKNLNDISTIIKSELTNGKALGLSHTYANVRINHVINLWGADFNAEGNLEAIYVTDSDSNASIGMKKYFVGVNKHGHVAISAKKIEGENIGAQVLGLFTLSTGQDSWQKLN

SEQ ID NO: 23 - protein, 312 aa, Streptococcus pyogenes
DDYQRNATEAYAKEVPHQITSVWTKGVTPLTPEQFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKQELTEGKALGLSHTYANVRINHVINLWGADFDAEGNLKAIYVTDSDSNASIGMKKYFVGVNAAGKVAISAKKIEGENIGAQVLGLFTLSTGQDSWNQTS

SEQ ID NO: 24 - protein, 312 aa, Streptococcus pyogenes
DDYQRNATEAYAKEVPHQITSVWTKGVTPLTPEQFTQGEDVFHAPYVANQGWYDITKTFNGKDDLLCGAATAGNMLHWWFDQNKDQIKRYLEEHPEKQKINFRGEQMFDVKEAIRTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKEELTKGKALGLSHTYANVRINHVINLWGADFDAEGNLKAIYVTDSDSNASIGMKKYFVGVNKAGKVAISAKKIEGENIGAQVLGLFTLSTGQDSWNQTN

SEQ ID NO: 25 - protein, 310 aa, Streptococcus pyogenes
DDYQRNATEAYAKEVPHQITSVWTKGVTPPEQFTQGEDVIHAPYVANQGWYDITKAFDGKDNLLCGAATAGNMLHWWFDQNKDQIKRYLEEHPEKQKINFRGEQMFDVKKAIDTKNHQLDSKLFNYFKEKAFPGLSARRIGVFPDHVIDMFINGYRLSLTNHGPTPVKEGSKDPRGGIFDAVFTRGNQSKLLTSRHDFKNKNLNDISTIIKQELTKGKALGLSHTYANVSINHVINLWGADFNAEGNLEAIYVTDSDSNASIGMKKYFVGVNAHGHVAISAKKIEGENIGAQVLGLFTLSTGQDSWQKLS

SEQ ID NO: 26 - protein, 320 aa, Streptococcus pyogenes
MDSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTNGGGHHHHHH

SEQ ID NO: 27 - protein, 325 aa, Streptococcus pyogenes
MDDYQRNATEAYAKEVPHQITSVWTKGVTPLTPEQFRYNNEDVIHAPYLAHQGWYDITKAFDGKDNLLCGAATAGNMLHWWFDQNKTEIEAYLSKHPEKQKIIFNNQELFDLKAAIDTKDSQTNSQLFNYFRDKAFPNLSARQLGVMPDLVLDMFINGYYLNVFKTQSTDVNRPYQDKDKRGGIFDAVFTRGDQTTLLTARHDLKNKGLNDISTIIKQELTEGRALALSHTYANVSISHVINLWGADFNAEGNLEAIYVTDSDANASIGMKKYFVGINAHGHVAISAKKIEGENIGAQVLGLFTLSSGKDIWQKLSGGGHHHHHH

SEQ ID NO: 28 - protein, 323 aa, Streptococcus pyogenes
MDDYQRNATEAYAKEVPHQITSVWTKGVTPLTPEQFRYNNEDVFHAPYVANQGWYDITKAFDGKDNLLCGAATAGNMLHWWFDQNKDQIKRYLEEHPEKQKINFNGDNMFDVKKAIDTKNHQLDSKLFNYFKEKAFPGLSARRIGVFPDHVIDMFINGYRLSLTNHGPTPVKEGSKDPRGGIFDAVFTRGNQSKLLTSRHDFKNKNLNDISTIIKQELTKGKALGLSHTYANVSINHVINLWGADFNAEGNLEAIYVTDSDSNASIGMKKYFVGVNAHGHVAISAKKIEGENIGAQVLGLFTLSTGQDSWQKLSGGGHHHHHH

SEQ ID NO: 29 - protein, 11 aa, Streptococcus pyogenes
PLTPEQFRYNN

SEQ ID NO: 30 - protein, 8 aa, Streptococcus pyogenes
PPANFTQG

SEQ ID NO: 31 - protein, 20 aa, Streptococcus pyogenes
DDYQRNATEAYAKEVPHQIT

SEQ ID NO: 32 - protein, 20 aa, Streptococcus pyogenes
DSFSANQEIRYSEVTPYHVT

SEQ ID NO: 33 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttggaagagcatccagaaaagcaaaaaataaacttcaatggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtgatcaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 34 - DNA, 978 bp, Streptococcus pyogenes
atggacgattaccaaaggaatgctacggaagcttatgccaaagaagtaccacatcagatcacttctgtatggaccaaaggtgttacaccactaacacccgagcagtttcgatataataacgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcatttttaacaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggagatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 35 - DNA, 972 bp, Streptococcus pyogenes
atggatgattatcagcgcaacgcgaccgaagcgtatgcgaaagaagtgccgcatcagattaccagcgtgtggaccaaaggcgtgaccccgctgaccccggaacagtttcgctataacaacgaagatgtgtttcatgcgccgtatgtggcgaaccagggctggtatgatattaccaaagcgtttgatggcaaagataacctgctgtgcggcgcggcgaccgcgggcaacatgctgcattggtggtttgatcagaacaaagatcagattaaacgctatctggaagaacatccggaaaaacagaaaattaactttaacggcgataacatgtttgatgtgaaaaaagcgattgataccaaaaaccatcagctggatagcaaactgtttaactattttaaagaaaaagcgtttccgggcctgagcgcgcgccgcattggcgtgtttccggatcatgtgattgatatgtttattaacggctatcgcctgagcctgaccaaccatggcccgaccccggtgaaagaaggcagcaaagatccgcgcggcggcatttttgatgcggtgtttacccgcggcaaccagagcaaactgctgaccagccgccatgattttaaaaacaaaaacctgaacgatattagcaccattattaaacaggaactgaccaaaggcaaagcgctgggcctgagccatacctatgcgaacgtgagcattaaccatgtgattaacctgtggggcgcggattttaacgcggaaggcaacctggaagcgatttatgtgaccgatagcgatagcaacgcgagcattggcatgaaaaaatattttgtgggcgtgaacgcgcatggccatgtggcgattagcgcgaaaaaaattgaaggcgaaaacattggcgcgcaggtgctgggcctgtttaccctgagcaccggccaggatagctggcagaaactgagcggcggtggccatcatcaccatcaccactaa

SEQ ID NO: 36 - DNA, 978 bp, Streptococcus pyogenes
atggatgattatcagcgcaacgcgaccgaagcgtatgcgaaagaagtgccgcatcagattaccagcgtgtggaccaaaggcgtgaccccgctgaccccggaacagtttcgctataacaacgaagatgtgattcatgcgccgtatctggcgaaccagggctggtatgatattaccaaagcgtttgatggcaaagataacctgctgtgcggcgcggcgaccgcgggcaacatgctgcattggtggtttgatcagaacaaaaccgaaattgaagcgtatctgagcaaacatccggaaaaacagaaaattatttttcgcaaccaggaactgtttgatctgaaagaagcgattcgcaccaaagatagccagaccaacagccagctgtttgaatattttcgcgataaagcgtttccgtatctgagcgcgcgccagctgggcgtgatgccggatctggtgctggatatgtttattaacggctattatctgaacgtgtttaaaacccagagcaccgatgtgaaacgcccgtatcaggataaagataaacgcggcggcatttttgatgcggtgtttacccgcggcaaccagaccaccctgctgaccgcgcgccatgatctgaaaaacaaaggcctgaacgatattagcaccattattaaagaagaactgaccaaaggccgcgcgctggcgctgagccatacctatgcgaacgtgagcattagccatgtgattaacctgtggggcgcggattttaacgcggaaggcaacctggaagcgatttatgtgaccgatagcgatgcgaacgcgagcattggcatgaaaaaatattttgtgggcattaacaaacatggccatgtggcgattagcgcgaaaaaaattgaaggcgaaaacattggcgcgcaggtgctgggcctgtttaccctgagcagcggcaaagatatttggcagaaactgaacggcggtggccatcatcaccatcaccactaa

SEQ ID NO: 37 - DNA, 978 bp, Streptococcus pyogenes
atggatgattatcagcgcaacgcgaccgaagcgtatgcgaaagaagtgccgcatcagattaccagcgtgtggaccaaaggcgtgaccccgctgaccccggaacagtttcgctataacaacgaagatgtgattcatgcgccgtatctggcgcatcagggctggtatgatattaccaaaacctttaacggcaaagataacctgctgtgcggcgcggcgaccgcgggcaacatgctgcattggtggtttgatcagaacaaaaccgaaattgaagcgtatctgagcaaacatccggaaaaacagaaaattatttttaacaacgaagaactgtttgatctgaaagcggcgattgataccaaagatagccagaccaacagccagctgtttaactattttaaagaaaaagcgtttccgaacctgagcacccgccagctgggcgtgatgccggatctggtgctggatatgtttattaacggctattatctgaacgtgtttaaaacccagagcaccgatgtgaaccgcccgtatcaggataaagataaacgcggcggcatttttgatgcggtgtttacccgcggcaaccagaccaccctgctgaccgcgcgccatgattttaaagaaaaaggcctgaaagatattagcaccattattaaacaggaactgaccgaaggccgcgcgctggcgctgagccatacctatgcgaacgtgagcattagccatgtgattaacctgtggggcgcggattttgatgcggaaggcaacctgaaagcgatttatgtgaccgatagcgatgcgaacgcgagcattggcatgaaaaaatattttgtgggcattaacgcgcatggcaaagtggcgattagcgcgaaaaaaattgaaggcgaaaacattggcgcgcaggtgctgggcctgtttaccctgagcagcggcaaagatatttggcagcagctgagcggcggtggccatcatcaccatcaccactaa

SEQ ID NO: 38 - DNA, 978 bp, Streptococcus pyogenes
atggatagctttagcgcgaaccaggaaattcgctatagcgaagtgaccccgtatcatgtgaccagcgtgtggaccaaaggcgtgaccccgctgaccccggaacagtttcgctataacaacgaagatgtgattcatgcgccgtatctggcgcatcagggctggtatgatattaccaaagcgtttgatggcaaagataacctgctgtgcggcgcggcgaccgcgggcaacatgctgcattggtggtttgatcagaacaaaaccgaaattgaagcgtatctgagcaaacatccggaaaaacagaaaattatttttaacaaccaggaactgtttgatctgaaagcggcgattgataccaaagatagccagaccaacagccagctgtttaactattttcgcgataaagcgtttccgaacctgagcgcgcgccagctgggcgtgatgccggatctggtgctggatatgtttattaacggctattatctgaacgtgtttaaaacccagagcaccgatgtgaaccgcccgtatcaggataaagataaacgcggcggcatttttgatgcggtgtttacccgcggcgatcagaccaccctgctgaccgcgcgccatgatctgaaaaacaaaggcctgaacgatattagcaccattattaaacaggaactgaccgaaggccgcgcgctggcgctgagccatacctatgcgaacgtgagcattagccatgtgattaacctgtggggcgcggattttaacgcggaaggcaacctggaagcgatttatgtgaccgatagcgatgcgaacgcgagcattggcatgaaaaaatattttgtgggcattaacgcgcatggccatgtggcgattagcgcgaaaaaaattgaaggcgaaaacattggcgcgcaggtgctgggcctgtttaccctgagcagcggcaaagatatttggcagaaactgagcggcggtggccatcatcaccatcaccactaa

SEQ ID NO: 39 - DNA, 918 bp, Streptococcus pyogenes
atgagcgtgtggaccaaaggcgtgaccccgctgaccccggaacagtttcgctataacaacgaagatgtgattcatgcgccgtatctggcgcatcagggctggtatgatattaccaaagcgtttgatggcaaagataacctgctgtgcggcgcggcgaccgcgggcaacatgctgcattggtggtttgatcagaacaaaaccgaaattgaagcgtatctgagcaaacatccggaaaaacagaaaattatttttaacaaccaggaactgtttgatctgaaagcggcgattgataccaaagatagccagaccaacagccagctgtttaactattttcgcgataaagcgtttccgaacctgagcgcgcgccagctgggcgtgatgccggatctggtgctggatatgtttattaacggctattatctgaacgtgtttaaaacccagagcaccgatgtgaaccgcccgtatcaggataaagataaacgcggcggcatttttgatgcggtgtttacccgcggcgatcagaccaccctgctgaccgcgcgccatgatctgaaaaacaaaggcctgaacgatattagcaccattattaaacaggaactgaccgaaggccgcgcgctggcgctgagccatacctatgcgaacgtgagcattagccatgtgattaacctgtggggcgcggattttaacgcggaaggcaacctggaagcgatttatgtgaccgatagcgatgcgaacgcgagcattggcatgaaaaaatattttgtgggcattaacgcgcatggccatgtggcgattagcgcgaaaaaaattgaaggcgaaaacattggcgcgcaggtgctgggcctgtttaccctgagcagcggcaaagatatttggcagaaactgagcggcggtggccatcatcaccatcaccactaa

SEQ ID NO: 40 - DNA, 975 bp, Streptococcus pyogenes
atggacgattaccaaaggaatgctacggaagcttatgccaaagaagtaccacatcagatcacttctgtatggaccaaaggtgttacaccactaacacccgagcagtttactcaaggtgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcatttttaacaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggagatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 41 - DNA, 969 bp, Streptococcus pyogenes
atggacgattaccaaaggaatgctacggaagcttatgccaaagaagtaccacatcagatcacttctgtatggaccaaaggtgttacaccacccgagcagtttactcaaggtgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcatttttaacaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggagatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 42 - DNA, 972 bp, Streptococcus pyogenes
atggacgattaccaaaggaatgctacggaagcttatgccaaagaagtaccacatcagatcacttctgtatggaccaaaggtgttacaccacccgagcagtttcgatataataacgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcatttttaacaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggagatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 43 - DNA, 969 bp, Streptococcus pyogenes
atggacgattaccaaaggaatgctacggaagcttatgccaaagaagtaccacatcagatcacttctgtatggaccaaaggtgttacaccacccgagcagtttactcaaggtgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcattattaacaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggagatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 44 - DNA, 969 bp, Streptococcus pyogenes
atggacgattaccaaaggaatgctacggaagcttatgccaaagaagtaccacatcagatcacttctgtatggaccaaaggtgttacaccacccgagcagtttactcaaggtgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcatttttcgtaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggagatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 45 - DNA, 969 bp, Streptococcus pyogenes
atggacgattaccaaaggaatgctacggaagcttatgccaaagaagtaccacatcagatcacttctgtatggaccaaaggtgttacaccacccgagcagtttactcaaggtgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcattattcgtaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggagatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 46 - DNA, 969 bp, Streptococcus pyogenes
atggatgattatcagcgcaacgcgaccgaagcgtatgcgaaagaagtgccgcatcagattaccagcgtgtggaccaaaggcgtgaccccgccggaacagtttactcaaggtgaagatgtgattcatgcgccgtatctggcgaaccagggctggtatgatattaccaaagcgtttgatggcaaagataacctgctgtgcggcgcggcgaccgcgggcaacatgctgcattggtggtttgatcagaacaaaaccgaaattgaagcgtatctgagcaaacatccggaaaaacagaaaattatttttcgcaaccaggaactgtttgatctgaaagaagcgattcgcaccaaagatagccagaccaacagccagctgtttgaatattttcgcgataaagcgtttccgtatctgagcgcgcgccagctgggcgtgatgccggatctggtgctggatatgtttattaacggctattatctgaacgtgtttaaaacccagagcaccgatgtgaaacgcccgtatcaggataaagataaacgcggcggcatttttgatgcggtgtttacccgcggcaaccagaccaccctgctgaccgcgcgccatgatctgaaaaacaaaggcctgaacgatattagcaccattattaaagaagaactgaccaaaggccgcgcgctggcgctgagccatacctatgcgaacgtgagcattagccatgtgattaacctgtggggcgcggattttaacgcggaaggcaacctggaagcgatttatgtgaccgatagcgatgcgaacgcgagcattggcatgaaaaaatattttgtgggcattaacaaacatggccatgtggcgattagcgcgaaaaaaattgaaggcgaaaacattggcgcgcaggtgctgggcctgtttaccctgagcagcggcaaagatatttggcagaaactgaacggcggtggccatcatcaccatcaccactaa

SEQ ID NO: 47 - DNA, 909 bp, Streptococcus pyogenes
atgtctgtatggaccaaaggtgttacaccacccgagcagtttactcaaggtgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcatttttcgtaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggaaaccagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 48 - DNA, 969 bp, Streptococcus pyogenes
atggacgattaccaaaggaatgctacggaagcttatgccaaagaagtaccacatcagatcacttctgtatggaccaaaggtgttacaccacccgagcagtttactcaaggtgaagatgtgatccatgcgccatatcttgctcatcaaggctggtacgatatcaccaaggccttcgatggggcggataatctcttgtgtggcgcagcaacggcaggtaatatgctgcattggtggtttgatcaaaataaaacagagattgaagcctatttaagtaaacaccctgaaaagcaaaaaatcatttttcgtaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggaaatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 49 - DNA, 909 bp, Streptococcus pyogenes
atgtctgtatggaccaaaggtgttacaccacccgagcagtttactcaaggtgaagatgtgatccatgcgccatatcttgctcatcaaggctggtacgatatcaccaaggccttcgatggggcggataatctcttgtgtggcgcagcaacggcaggtaatatgctgcattggtggtttgatcaaaataaaacagagattgaagcctatttaagtaaacaccctgaaaagcaaaaaatcatttttcgtaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggaaatcagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 50 - DNA, 969 bp, Streptococcus pyogenes
atggacgattaccaaaggaatgctacggaagcttatgccaaagaagtaccacatcagatcacttctgtatggaccaaaggtgttacaccacccgagcagtttactcaaggtgaagatgtgatccatgcgccatatcttgctcatcaaggctggtacgatatcaccaaggccttcgatgggaaggataatctcttgtgtggcgcagcaacggcaggtaatatgctgcattggtggtttgatcaaaataaaacagagattgaagcctatttaagtaaacaccctgaaaagcaaaaaatcatttttcgtaaccaagagctatttgatttgaaagctgctatcgataccaaggacagtcaaaccaatagtcagctttttaattattttagagataaagcctttccaaatctatcagcacgtcaactcggggttatgcctgatcttgttctagatatgtttatcaatggttactacttaaatgtgtttaaaacacagtctactgatgtcaatcgaccttatcaggacaaggacaaacgaggtggtattttcgatgctgttttcaccagaggaaaccagacaacgctcttgacagctcgtcatgatttaaaaaataaaggactaaatgacatcagcaccattatcaagcaagaactgactgaaggaagagcccttgctttatcacatacctacgccaatgttagcattagccatgtgattaacttgtggggagctgattttaatgctgaaggaaaccttgaggccatctatgtcacagactcagatgctaatgcgtctattggtatgaaaaaatattttgtcggcattaatgctcatggacatgtcgccatttctgccaagaaaatagaaggagaaaacattggcgctcaagtattaggcttatttacgctttccagtggcaaggacatttggcagaaactgagcggcggtggccatcatcaccatcaccactaa

SEQ ID NO: 51 - DNA, 969 bp, Streptococcus pyogenes
atggatgattatcagcgcaacgcgaccgaagcgtatgcgaaagaagtgccgcatcagattaccagcgtgtggaccaaaggcgtgaccccgctgaccccggaacagtttacccagggcgaagatgtgtttcatgcgccgtatgtggcgaaccagggctggtatgatattaccaaagcgtttgatggcaaagataacctgctgtgcggcgcggcgaccgcgggcaacatgctgcattggtggtttgatcagaacaaagatcagattaaacgctatctggaagaacatccggaaaaacagaaaattaactttaacggcgaaaacatgtttgatgtgaaaaaagcgattgataccaaaaaccatcagctggatagcaaactgtttaactattttaaagaaaaagcgtttccgtatctgagcgcgaaacatctgggcgtgtttccggatcatgtgattgatatgtttattaacggctatcgcctgagcctgaccaaccatggcccgaccccggtgaaagaaggcagcaaagatccgcgcggcggcatttttgatgcggtgtttacccgcggcaaccagagcaaactgctgaccagccgccatgattttaaaaacaaaaacctgaacgatattagcaccattattaaacaggaactgaccaaaggcaaagcgctgggcctgagccatacctatgcgaacgtgcgcattaaccatgtgattaacctgtggggcgcggattttaacgcggaaggcaacctggaagcgatttatgtgaccgatagcgatagcaacgcgagcattggcatgaaaaaatattttgtgggcgtgaacgcgcatggccatgtggcgattagcgcgaaaaaaattgaaggcgaaaacattggcgcgcaggtgctgggcctgtttaccctgagcaccggccaggatagctggcagaaactgagcggcggtggccatcatcaccatcaccactaa

SEQ ID NO: 52 - DNA, 969 bp, Streptococcus pyogenes
atggatgattatcagcgcaacgcgaccgaagcgtatgcgaaagaagtgccgcatcagattaccagcgtgtggaccaaaggcgtgaccccgctgaccccggaacagtttacccagggcgaagatgtgtttcatgcgccgtatgtggcgaaccagggctggtatgatattaccaaagcgtttgatggcaaagataacctgctgtgcggcgcggcgaccgcgggcaacatgctgcattggtggtttgatcagaacaaagatcagattaaacgctatctggaagaacatccggaaaaacagaaaattaactttcgcggcgaaaacatgtttgatgtgaaagaagcgattcgcaccaaaaaccatcagctggatagcaaactgtttgaatattttaaagaaaaagcgtttccgtatctgagcgcgaaacatctgggcgtgtttccggatcatgtgattgatatgtttattaacggctatcgcctgagcctgaccaaccatggcccgaccccggtgaaaaaaggcagcaaagatccgcgcggcggcatttttgatgcggtgtttacccgcggcaaccagagcaaactgctgaccagccgccatgattttaaaaacaaaaacctgaacgatattagcaccattattaaaagcgaactgaccaacggcaaagcgctgggcctgagccatacctatgcgaacgtgcgcattaaccatgtgattaacctgtggggcgcggattttaacgcggaaggcaacctggaagcgatttatgtgaccgatagcgatagcaacgcgagcattggcatgaaaaaatattttgtgggcgtgaacaaacatggccatgtggcgattagcgcgaaaaaaattgaaggcgaaaacattggcgcgcaggtgctgggcctgtttaccctgagcaccggccaggatagctggcagaaactgaacggcggtggccatcatcaccatcaccactaa

SEQ ID NO: 53 - DNA, 969 bp, Streptococcus pyogenes
atggatgattatcagcgcaacgcgaccgaagcgtatgcgaaagaagtgccgcatcagattaccagcgtgtggaccaaaggcgtgaccccgctgaccccggaacagtttacccagggcgaagatgtgtttcatgcgccgtatgtggcgaaccagggctggtatgatattaccaaaacctttaacggcaaagatgatctgctgtgcggcgcggcgaccgcgggcaacatgctgcattggtggtttgatcagaacaaagatcagattaaacgctatctggaagaacatccggaaaaacagaaaattaactttaacggcgaacagatgtttgatgtgaaagaagcgattgataccaaaaaccatcagctggatagcaaactgtttgaatattttaaagaaaaagcgtttccgtatctgagcaccaaacatctgggcgtgtttccggatcatgtgattgatatgtttattaacggctatcgcctgagcctgaccaaccatggcccgaccccggtgaaagaaggcagcaaagatccgcgcggcggcatttttgatgcggtgtttacccgcggcaaccagagcaaactgctgaccagccgccatgattttaaagaaaaaaacctgaaagaaattagcgatctgattaaacaggaactgaccgaaggcaaagcgctgggcctgagccatacctatgcgaacgtgcgcattaaccatgtgattaacctgtggggcgcggattttgatgcggaaggcaacctgaaagcgatttatgtgaccgatagcgatagcaacgcgagcattggcatgaaaaaatattttgtgggcgtgaacgcggcgggcaaagtggcgattagcgcgaaaaaaattgaaggcgaaaacattggcgcgcaggtgctgggcctgtttaccctgagcaccggccaggatagctggaaccagaccagcggcggtggccatcatcaccatcaccactaa

SEQ ID NO: 54 - DNA, 969 bp, Streptococcus pyogenes
atggatgattatcagcgcaacgcgaccgaagcgtatgcgaaagaagtgccgcatcagattaccagcgtgtggaccaaaggcgtgaccccgctgaccccggaacagtttacccagggcgaagatgtgtttcatgcgccgtatgtggcgaaccagggctggtatgatattaccaaaacctttaacggcaaagatgatctgctgtgcggcgcggcgaccgcgggcaacatgctgcattggtggtttgatcagaacaaagatcagattaaacgctatctggaagaacatccggaaaaacagaaaattaactttcgcggcgaacagatgtttgatgtgaaagaagcgattcgcaccaaaaaccatcagctggatagcaaactgtttgaatattttaaagaaaaagcgtttccgtatctgagcaccaaacatctgggcgtgtttccggatcatgtgattgatatgtttattaacggctatcgcctgagcctgaccaaccatggcccgaccccggtgaaaaaaggcagcaaagatccgcgcggcggcatttttgatgcggtgtttacccgcggcaaccagagcaaactgctgaccagccgccatgattttaaagaaaaaaacctgaaagaaattagcgatctgattaaagaagaactgaccaaaggcaaagcgctgggcctgagccatacctatgcgaacgtgcgcattaaccatgtgattaacctgtggggcgcggattttgatgcggaaggcaacctgaaagcgatttatgtgaccgatagcgatagcaacgcgagcattggcatgaaaaaatattttgtgggcgtgaacaaagcgggcaaagtggcgattagcgcgaaaaaaattgaaggcgaaaacattggcgcgcaggtgctgggcctgtttaccctgagcaccggccaggatagctggaaccagaccaacggcggtggccatcatcaccatcaccactaa

SEQ ID NO: 55 - DNA, 963 bp, Streptococcus pyogenes
atggatgattatcagcgcaacgcgaccgaagcgtatgcgaaagaagtgccgcatcagattaccagcgtgtggaccaaaggcgtgaccccgccggaacagtttactcaaggtgaagatgtgattcatgcgccgtatgtggcgaaccagggctggtatgatattaccaaagcgtttgatggcaaagataacctgctgtgcggcgcggcgaccgcgggcaacatgctgcattggtggtttgatcagaacaaagatcagattaaacgctatctggaagaacatccggaaaaacagaaaattaactttcgcggcgaacagatgtttgatgtgaaaaaagcgattgataccaaaaaccatcagctggatagcaaactgtttaactattttaaagaaaaagcgtttccgggcctgagcgcgcgccgcattggcgtgtttccggatcatgtgattgatatgtttattaacggctatcgcctgagcctgaccaaccatggcccgaccccggtgaaagaaggcagcaaagatccgcgcggcggcatttttgatgcggtgtttacccgcggcaaccagagcaaactgctgaccagccgccatgattttaaaaacaaaaacctgaacgatattagcaccattattaaacaggaactgaccaaaggcaaagcgctgggcctgagccatacctatgcgaacgtgagcattaaccatgtgattaacctgtggggcgcggattttaacgcggaaggcaacctggaagcgatttatgtgaccgatagcgatagcaacgcgagcattggcatgaaaaaatattttgtgggcgtgaacgcgcatggccatgtggcgattagcgcgaaaaaaattgaaggcgaaaacattggcgcgcaggtgctgggcctgtttaccctgagcaccggccaggatagctggcagaaactgagcggcggtggccatcatcaccatcaccactaa

SEQ ID NO: 56 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 57 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFKGEQMFDVKEAIDTKNHQLDSKLFEYFKEKAFPYLSTKHLGVFPDHVIDMFINGYRLSLTNHGPTPVKRGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 58 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 59 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLREHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELDEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 60 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFNGEQMFDVKEAIRTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELEEGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 61 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIERYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEKNIGAQVLGLFTLSTGQKSWNQTN

SEQ ID NO: 62 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFRGEQMFDVKEAIRTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKSELENGKALGLSHTYANVRINHVINLWGADFDSNGNLKAIYVTDSDSNASIGMKKYFVGVNKAGKVAISAKEIKEDNIGAQVLGLFTLSTGQDSWNQTN

SEQ ID NO: 63 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLKEHPEKQKINFRGEQMFDVKEAIRTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKKELEEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 64 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIERYLEEHPEKQKINFRGEQMFDVKEAIDTKNHQLDSKLFEYFKEKAFPYLSTKHLGVFPDHVIDMFINGYRLSLTNHGPTPVKKGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTN

SEQ ID NO: 65 - protein, 306 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSW

SEQ ID NO: 66 - protein, 290 aa, Streptococcus pyogenes
SVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 67 - protein, 290 aa, Streptococcus pyogenes
SVWTKGVTPPANFTQGEDVFHAPYVANQGWYDITKTFNGKDDLLCGAATAGNMLHWWFDQNKDQIKRYLEEHPEKQKINFKGEQMFDVKEAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 68 - protein, 290 aa, Streptococcus pyogenes
SVWTKGVTPPANFTQGEDVFHAPYVANQGWYDITKTFNGKDDLLCGAATAGNMLHWWFDQNKDQIERYLEEHPEKQKINFKGEQMFDVKKAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTN

SEQ ID NO: 69 - protein, 319 aa, Streptococcus pyogenes
DDYQRNATEAYAKEVPHQITSVWTKGVTPPANFTQGEDVFHAPYVANQGWYDITKTFNGKDDLLCGAATAGNMLHWWFDQNKDQIERYLEEHPEKQKINFKGEQMFDVKKAIDTKNHQLDSKLFEYFKEKAFPYLSTKHLGVFPDHVIDMFINGYRLSLTNHGPTPVKEGSKDPRGGIFDAVFTRGNQSKLLTSRHDFKEKNLKEISDLIKEELTKGKALGLSHTYANVRINHVINLWGADFDSNGNLKAIYVTDSDSNASIGMKKYFVGVNSAGKVAISAKEIKEDNIGAQVLGLFTLSTGQKSWNQTNGGGHHHHHH

SEQ ID NO: 70 - protein, 4 aa, Streptococcus pyogenes
NQTN

SEQ ID NO: 71 - protein, 20 aa, Streptococcus pyogenes
DSFSANQEIRYSEVTPYHVT

SEQ ID NO: 72 - DNA, 963 bp, Streptococcus pyogenes; identical to SEQ ID NO: 33

SEQ ID NO: 73 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttggaagagcatccagaaaagcaaaaaataaacttccgtggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtgatcaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 74 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttggaagagcatccagaaaagcaaaaaataaacttcaaaggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaacgtggtagtaaagatccccgaggtggtatttttgacgccgtatttacaagaggtaaccaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 75 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttggaagagcatccagaaaagcaaaaaataaacttccgtggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaaaaaggtagtaaagatccccgaggtggtatttttgacgccgtatttacaagaggtaaccaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaataaagctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 76 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttgcgtgagcatccagaaaagcaaaaaataaacttcaatggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtaaccaaagtaagctattgacaagtcgtcatgattttaaagaaaaaaatctcaaagaaatcagtgatctcattaagaaagagttagatgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 77 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttgaaagagcatccagaaaagcaaaaaataaacttcaatggcgaacagatgtttgacgtaaaagaagctatccgtactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtaaccaaagtaagctattgacaagtcgtcatgattttaaagaaaaaaatctcaaagaaatcagtgatctcattaagaaagagttagaagaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaataaagctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 78 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattgaacgttatttggaagagcatccagaaaagcaaaaaataaacttcaatggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtaaccaaagtaagctattgacaagtcgtcatgattttaaagaaaaaaatctcaaagaaatcagtgatctcattaaggaagagttaaccaaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaaaaaaatataggtgctcaagtactagggttatttacactttcaacagggcaaaaaagttggaatcagaccaatggcggtggccatcatcaccatcaccactaa

SEQ ID NO: 79 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttgaaagagcatccagaaaagcaaaaaataaacttccgtggcgaacagatgtttgacgtaaaagaagctatccgtactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtaaccaaagtaagctattgacaagtcgtcatgattttaaagaaaaaaatctcaaagaaatcagtgatctcattaagagtgagttagaaaacggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaataaagctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 80 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttgaaagagcatccagaaaagcaaaaaataaacttccgtggcgaacagatgtttgacgtaaaagaagctatccgtactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaaaaaggtagtaaagatccccgaggtggtatttttgacgccgtatttacaagaggtaaccaaagtaagctattgacaagtcgtcatgattttaaagaaaaaaatctcaaagaaatcagtgatctcattaagaaagagttagaagaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 81 - DNA, 963 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattgaacgttatttggaagagcatccagaaaagcaaaaaataaacttccgtggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaaaaaggtagtaaagatccccgaggtggtatttttgacgccgtatttacaagaggtaaccaaagtaagctattgacaagtcgtcatgattttaaagaaaaaaatctcaaagaaatcagtgatctcattaaggaagagttaaccaaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaaaaaagttggaatcagaccaatggcggtggccatcatcaccatcaccactaa

SEQ ID NO: 82 - DNA, 957 bp, Streptococcus pyogenes
atggatagtttttctgctaatcaagagattagatattcggaagtaacaccttatcacgttacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttggaagagcatccagaaaagcaaaaaataaacttcaatggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtgatcaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggggtggcggcggtggccatcatcaccatcaccactaa

SEQ ID NO: 83 - DNA, 903 bp, Streptococcus pyogenes
atgtccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttggaagagcatccagaaaagcaaaaaataaacttcaatggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtgatcaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 84 - DNA, 903 bp, Streptococcus pyogenes
atgtccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattaaacgttatttggaagagcatccagaaaagcaaaaaataaacttcaaaggcgaacagatgtttgacgtaaaagaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtaaccaaagtaagctattgacaagtcgtcatgattttaaagaaaaaaatctcaaagaaatcagtgatctcattaagaaagagttaaccgaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaagatagttggaatcagaccaatggcggtggccatcatcaccatcaccactaa

SEQ ID NO: 85 - DNA, 903 bp, Streptococcus pyogenes
atgtccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattgaacgttatttggaagagcatccagaaaagcaaaaaataaacttcaaaggcgaacagatgtttgacgtaaaaaaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtaaccaaagtaagctattgacaagtcgtcatgattttaaagaaaaaaatctcaaagaaatcagtgatctcattaaggaagagttaaccaaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaaaaaagttggaatcagaccaatggcggtggccatcatcaccatcaccactaa

SEQ ID NO: 86 - DNA, 963 bp, Streptococcus pyogenes
atggacgattaccaaaggaatgctacggaagcttatgccaaagaagtaccacatcagatcacttccgtttggaccaaaggagttactcctccagcaaacttcactcaaggtgaagatgtttttcacgctccttatgttgctaaccaaggatggtatgatattaccaaaacattcaatggaaaagacgatcttctttgcggggctgccacagcagggaatatgcttcactggtggttcgatcaaaacaaagaccaaattgaacgttatttggaagagcatccagaaaagcaaaaaataaacttcaaaggcgaacagatgtttgacgtaaaaaaagctatcgacactaaaaaccaccagctagatagtaaattatttgaatattttaaagaaaaagctttcccttatctatctactaaacacctaggagttttccctgatcatgtaattgatatgttcattaacggctaccgccttagtctaactaaccacggtccaacgccagtaaaagaaggtagtaaagatccccgaggtggtatttttgacgccgtatttacaagaggtaaccaaagtaagctattgacaagtcgtcatgattttaaagaaaaaaatctcaaagaaatcagtgatctcattaaggaagagttaaccaaaggcaaggctctaggcctatcacacacctacgctaacgtacgcatcaaccatgttataaacctgtggggagctgactttgattctaacgggaaccttaaagctatttatgtaacagactctgatagtaatgcatctattggtatgaagaaatactttgttggtgttaattccgctggaaaagtagctatttctgctaaagaaataaaagaagataatataggtgctcaagtactagggttatttacactttcaacagggcaaaaaagttggaatcagaccaatggcggtggccatcatcaccatcaccactaa

SEQ ID NO: 87 - protein, 19 aa, Streptococcus pyogenes
SFSANQEIRYSEVTPYHVT

SEQ ID NO: 88 - protein, 19 aa, Streptococcus pyogenes
DYQRNATEAYAKEVPHQIT

SEQ ID NO: 89 - protein, 20 aa, Streptococcus pyogenes
DDYQRNATEAYAKEVPHQIT

(19) United States
(12) Patent Application Publication
(10) Pub. No.: US 2023/0110085 A1
(43) Pub. Date: Apr. 13, 2023

(54) IMMUNOGLOBULIN DETECTION AND ASSOCIATED THERAPIES

(71) Applicant: Hansa Biopharma AB, Lund (SE)

(72) Inventors: Anna RUNSTRÖM, Höllviken (SE); Robert BOCKERMANN, Malmö (SE); Kristoffe SJÖHOLM, Lund (SE); Markus ROUPÉ, Lomma (SE); Christian KJELLMAN, Lövestad (SE); Tomas LORANT, Uppsala (SE)

(73) Assignee: Hansa Biopharma AB, Lund (SE)

(21) Appl. No.: 17/799,309

(22) PCT Filed: Feb. 12, 2021
(86) PCT No.: PCT/EP2021/053462

§ 371 Date: Aug. 12, 2022

(30) Foreign Application Priority Data
Feb. 14, 2020 (GB) 2002072.3

(51) Int. Cl. G01N 33/68; A61K 38/48

(52) U.S. Cl. CPC G01N 33/6854; A61K 38/4873; G01N 2333/96466

(57) ABSTRACT

The present invention relates to improved methods for detecting immunoglobulin M (IgM), immunoglobulin A (IgA) and immunoglobulin E (IgE) antibodies, and new therapies for diseases and conditions mediated by pathogenic antibodies and antibody complexes.

### FIELD OF THE INVENTION

The present invention relates to improved methods for detecting immunoglobulin M (IgM), immunoglobulin A (IgA) and immunoglobulin E (IgE) antibodies, and new therapies for diseases and conditions mediated by pathogenic antibodies and antibody complexes.

### BACKGROUND OF THE INVENTION

Autoimmune diseases and other conditions mediated by pathogenic endogenous antibodies present complex therapeutic challenges. The last option for patients suffering from, for example, kidney, liver or heart failure is often organ transplant, but human leukocyte antigen (HLA) sensitization is a major barrier to organ transplantation. Highly sensitized patients have high levels of anti-HLA antibodies, which are likely to target and significantly compromise a transplanted organ. The abundance of such antibodies directly affects the likelihood of finding a donor organ that will be a match. Many highly sensitized patients suffering from renal failure, for example, will indefinitely remain in a debilitating disease state on long-term dialysis, which is associated with a high cost, a poor quality of life and an increased mortality rate. Immunosuppressive drugs can reduce early graft loss due to acute rejection but they are less effective at addressing graft loss due to chronic rejection and they increase the risk of serious complications, such as life-threatening infections and cancers.

Pathogenic endogenous antibodies and immune complexes also cause a wide range of autoimmune diseases and conditions, whereby a patient has antibodies that recognise a self-antigen and these antibodies mediate inflammation and tissue damage.

Diagnosis and treatment of autoimmune diseases and other conditions mediated by pathogenic endogenous require sensitive assays for detecting pathogenic endogenous antibodies and therapeutic methods for reducing the deleterious effects of the antibodies. However, antibodies are complex molecules and current immunoassays for detecting them can be unreliable and insufficiently sensitive. Current methods for detecting IgM antibodies, such as anti-HLA IgM antibodies, are adapted from methods for detecting IgG antibodies (Paantjens, et al., Pulm. Med., 2011). If problems with interference are suspected in methods for detecting IgG, it is common to use a reducing agent such as dithiothreitol (DTT) to disrupt IgM and prevent IgM blocking IgG (Paantjens, et al., Pulm. Med., 2011). There is a requirement for improved assays for detecting antibodies, in particular methods for detecting IgM antibodies and further classes of antibodies other than IgG. New methods for treating patients with pathogenic antibodies are also required.

The immunoglobulin G-degrading cysteine protease, imlifidase (IdeS) is an IgG endopeptidase that is currently under development as a rapid desensitization treatment in kidney transplantation. Imlifidase is highly specific and cleaves all subclasses of human IgG. It has been suggested that imlifidase also cleaves pathological anti-HLA IgM (Zhang et al. 2019, Am J Transplant. 2019; 19 (suppl 3)).

### SUMMARY OF THE INVENTION

The invention provides an improved method for detecting IgM antibodies, IgA antibodies or IgE antibodies in a sample comprising contacting said sample with an IgG cysteine protease under conditions which permit IgG cysteine protease activity to occur, and contacting said sample with an agent suitable for detecting IgM antibodies, IgA antibodies or IgE antibodies.

In preferred embodiments, the method of the invention is for detecting IgM antibodies. The inventors have shown in the examples that IgM antibodies can form immune complexes with IgG that interfere with detection of antigen-specific IgM antibodies. These complexes can be cleaved by IgG cysteine proteases, so the use of an IgG cysteine protease will provide improvement to any method for detecting IgM antibodies.

In certain embodiments, the method of the invention is for detecting IgA antibodies. IgA antibodies are known to form immune complexes with IgG, in a similar manner to IgM antibodies. Such complexes may cause interference with any method of detecting antigen specific IgA antibodies, so the use of an IgG cysteine protease will provide improvement to any method for detecting IgA antibodies, as demonstrated for IgM antibodies in the examples.

In certain embodiments, the method of the invention is for detecting IgE antibodies. IgE antibodies are known to form immune complexes with IgG, in a similar manner to IgM antibodies. Such complexes may cause interference with any method of detecting antigen specific IgE antibodies, so the use of an IgG cysteine protease will provide improvement to any method for detecting IgE antibodies, as demonstrated for IgM antibodies in the examples.

In preferred embodiments, the IgG cysteine protease an IdeS or IdeZ polypeptide, such as a polypeptide having a sequence that is at least 80% identical to SEQ ID NO: 2, 4 or 5, such as at least 85%, 90%, 95% or 99% identical. These polypeptides have established specificity, confirmed in the Examples, for IgG and so are effective for eradicating IgG interference without affecting IgM, IgA or IgE levels.

In certain embodiments, said agent suitable for detecting IgM antibodies, IgA antibodies or IgE antibodies is an anti-IgM antibody, an anti-IgA antibody or an anti-IgE antibody. Such antibodies are readily available. The antibodies may be fixed to a solid substrate, such as a bead or a plate, to aid handling of samples and increase throughput. In certain embodiments, the agent suitable for detecting IgM antibodies, IgA antibodies or IgE antibodies is labelled, to aid detection.

The methods of the invention are particularly useful for detecting IgM antibodies, IgA antibodies or IgE antibodies against a specific-antigen. The Examples demonstrate that the IgM-IgG immune complexes that are cleaved by IgG cysteine proteases can provide false-positive results in assays for target-specific antibodies. Therefore, in certain embodiments, the sample is contacted with an antigen of interest under conditions that permit isolation of antibodies that bind specifically to the antigen of interest. The antigen of interest may be, for example, a Human Leukocyte Antigen (HLA), an erythrocyte antigen or a drug antigen.

The methods of the invention are particularly useful for analysing patient samples, such as serum samples, for example from a patient that has been diagnosed as requiring an organ transplant, or a patient diagnosed with a disease selected from Table A.

The invention also provides kits for practising the methods of the invention, comprising an IgG cysteine protease and an agent for detecting an IgM antibody, an IgA antibody or an IgE antibody, and optionally comprising an antigen of interest, such as a Human Leukocyte Antigen (HLA), an erythrocyte antigen or a drug antigen

The invention also provides a method of treating a disease or condition mediated in whole or in part by pathogenic IgM antibodies, pathogenic IgA antibodies or pathogenic IgE antibodies, wherein the method comprises administering an IgG cysteine protease. The inventors have demonstrated in the examples that IgM antibodies can form immune complexes with IgG antibodies that can be cleaved with an IgG cysteine protease. IgA and IgE antibodies will likely form similar complexes with IgG antibodies. These immune complexes are expected to mediate disease processes, so IgG cysteine proteases will be useful for treating diseases or conditions mediated by pathogenic IgM antibodies, pathogenic IgA antibodies or pathogenic IgE antibodies. The invention also provides an IgG cysteine protease, for use in a method of treating a disease or condition mediated in whole or in part by pathogenic IgM antibodies, pathogenic IgA antibodies or pathogenic IgE antibodies.

The invention also provides methods of treating in a subject a disease or condition mediated in whole or in part by pathogenic IgG antibodies, wherein said subject is determined to exhibit anti-IgG IgM antibodies, anti-IgG IgA antibodies or anti-IgG IgE antibodies, wherein the method comprises administering an IgG cysteine protease. The inventors have demonstrated in the examples that IgM antibodies can form immune complexes with IgG antibodies that can be cleaved with an IgG cysteine protease. IgA and IgE antibodies will likely form similar complexes with IgG antibodies. These immune complexes are expected to amplify the effects of pathogenic IgG antibodies, so an IgG cysteine protease will be especially effective at treating patients with anti-IgG IgM antibodies or anti-IgG IgA antibodies. In further preferred embodiments, the invention provides an IgG cysteine protease, for use in a method of treating in a subject a disease or condition mediated in whole or in part by pathogenic IgG antibodies, wherein said subject is determined to exhibit anti-IgG IgM antibodies, anti-IgG IgA antibodies or anti-IgG IgE antibodies.

The invention also provides methods of treating in a subject a disease or condition mediated in whole or in part by complexes of IgG antibodies and IgM, IgA or IgE antibodies, wherein the method comprises administering an IgG cysteine protease. The inventors have demonstrated in the examples that IgM antibodies can form immune complexes with IgG antibodies that can be cleaved with an IgG cysteine protease. IgA and IgE antibodies will likely form similar complexes with IgG antibodies. These immune complexes may mediate disease processes, so an IgG cysteine protease will be effective at treating diseases mediated by complexes of IgG antibodies and IgM, IgA or IgE antibodies. In further preferred embodiments, the invention provides an IgG cysteine protease, for use in a method of treating in a subject a disease or condition mediated in whole or in part by complexes of IgG antibodies and IgM, IgA or IgE antibodies.

### BRIEF DESCRIPTION OF THE FIGURES

FIG. 1—Schematic showing IgM and its disulphide bridges

FIG. 2—No cleavage of human IgM was seen after incubation with imlifidase

FIG. 3—Purification of IgG was successfully performed in serum using CaptureSelect IgG-CH1 Affinity Matrix

FIG. 4—Effect of imlifidase was seen on HLA I+II antibodies (IgG, HI), MFI cut-off>3000

FIG. 5—HLA I+II antibodies (IgG, EDTA) vs HLA I+II antibodies (IgM, EDTA)

FIG. 6—Comparison of effects seen on anti-HLA IgM antibodies in neat serum and after IgG depletion

FIG. 7—Detection limits of the assay—anti-human IgM (PE)

FIG. 8—Imlifidase does not digest human IgM in sensitized patients

FIG. 9—A) The relative intensity (MFI) of SAB-HLA class I and II specific IgM 24 hours post-imlifidase to pre-dose serum samples from sensitized patients with ESRD. Each point represents a SAB-HLA bead in a patient serum. The positive bead cut off is 500 in ether of the samples, only positive beads are included in the analysis. B) Beads that are increased in the 24 hours samples. C) Beads that are decreased in the 24 hours samples. For reference, IgG SAB-HLA from pre-dose serum is included in the graphs. Individual HLA beads are connected via lines. Individual patients are labelled with different point and line type.

FIG. 10—Digestion of purified human IgM and IgG were analysed on SDS-PAGE gel (4-20%). Purified human IgM (A) and IgG (B) were incubated with a wide range of imlifidase concentrations for 2 hours at 37° C. The high molecular weight IgM samples (A) were reduced with DTT and separated on an SDS-PAGE gel. IgG samples (B) were heated for 3 minutes and separated unreduced on an SDS-PAGE gel.

FIG. 11—Serum samples from 4 patients with ESRD were incubated with high concentrations of imlifidase. Digestion of human IgM was analysed by SDS-PAGE gel (A) and Western blot (B).

FIG. 12—SDS-PAGE evaluation of serum samples both before and after depletion of IgG. A) contains sample form Patient 02-922, Patient 02-923, Patient 02-925 and Patient 02-926. B) contains sample form Patient 02-927, Patient 02-928 and Patient 02-929.

FIG. 13—IgG depleted pre-dose and 24 hours post-imlifidase serum samples from sensitized patients with ESRD analysed for SAB-HLA class I and II (IgM). The N in the figure is the number of Single Antigen Beads reaching a threshold of 1000 MFI in the pre-dose sera (from analysis on IgM in neat sera [FIG. 4]) for that specific patient, only positive beads included in the analysis. A) Patient 02-922. B) Patient 02-923. C) Patient 02-925. D) Patient 02-926. E) Patient 02-927. F) Patient 02-928. G) Patient 02-929.

FIG. 14—Pre-dose and 24 hours post-imlifidase serum samples from one sensitized patient (02-927) with ESRD were compared with in vitro treatment with imlifidase for 1 hour incubation at 6 and 30 μg/mL. Serum samples were analysed for SAB-HLA class I and II (IgG and IgM).

FIG. 15—Our proposed mechanism is that IgG-DSA complexed IgM bind to the SAB-HLA beads. The IgM detection antibody recognizes IgM in the complex, resulting in artefactual IgM signal. After imlifidase treatment, these IgG-IgM complexes will be cleaved, and a much lower but true IgM signal will be recorded in the SAB HLA assay.

FIG. 16—Pre-dose and 24 hours post-imlifidase serum samples from sensitized patients with ESRD analysed for SAB-HLA class I and II specific IgG. For clarity only Single Antigen Beads reaching a threshold of 3000 MFI in the pre-dose sera are included in the analysis. N indicates the number of positive beads of patient 02-922 (A), 02-923 (B), 02-925 (C), 02-926 (D), 02-927 (E), 02-928 (F) and 02-929 (G).

FIG. 17—Beads that varies a small amount between pre and post dose for IgM. Lines and shape are according to legend.

### BRIEF DESCRIPTION OF THE SEQUENCES

SEQ ID NO: 1 is the full sequence of IdeS including N terminal methionine and signal sequence. It is also available as NCBI Reference sequence no. WP_010922160.1

SEQ ID NO: 2 is the mature sequence of IdeS, lacking the N terminal methionine and signal sequence. It is also available as Genbank accession no. ADF13949.1

SEQ ID NO: 3 is the full sequence of IdeZ including N terminal methionine and signal sequence. It is also available as NCBI Reference sequence no. WP_014622780.1.

SEQ ID NO: 4 is the mature sequence of IdeZ, lacking the N terminal methionine and signal sequence.

SEQ ID NO: 5 is the sequence of a hybrid IdeS/Z. The N terminus is based on IdeZ lacking the N terminal methionine and signal sequence.

SEQ ID NOs: 6 to 25 are the sequences of exemplary proteases for use in the methods of the invention.

SEQ ID NO: 26 is the sequence of an IdeS polypeptide. Comprises the sequence of SEQ ID NO: 2 with an additional N terminal methionine and a histidine tag (internal reference pCART124).

SEQ ID NO: 27 is the sequence of an IdeZ polypeptide. Comprises the sequence of SEQ ID NO: 4 with an additional N terminal methionine and a histidine tag (internal reference pCART144).

SEQ ID NO: 28 is the sequence of an IdeS/Z polypeptide. Comprises the sequence of SEQ ID NO: 5 with an additional N terminal methionine and a histidine tag (internal reference pCART145).

SEQ ID NO: 29 is the contiguous sequence PLTPEQFRYNN, which corresponds to positions 63-73 of SEQ ID NO: 3.

SEQ ID NO: 30 is the contiguous sequence PPANFTQG, which corresponds to positions 58-65 of SEQ ID NO: 1.

SEQ ID NO: 31 is the contiguous sequence DDYQRNATEAYAKEVPHQIT, which corresponds to positions 35-54 of SEQ ID NO: 3.

SEQ ID NO: 32 is the contiguous sequence DSFSANQEIRYSEVTPYHVT, which corresponds to positions 30-49 of SEQ ID NO: 1.

SEQ ID NOs: 33 to 55 are nucleotide sequences encoding proteases set out above.

SEQ ID NOs: 56 to 69 are the sequences of exemplary proteases for use in the methods of the invention.

SEQ ID NO: 70 is the contiguous sequence NQTN, which corresponds to positions 336-339 of SEQ ID NO: 1.

SEQ ID NO: 71 is the contiguous sequence DSFSANQEIR YSEVTPYHVT, which corresponds to positions 30-49 of SEQ ID NO: 1.

SEQ ID NOs: 72 to 86 are nucleotide sequences encoding polypeptides disclosed herein.

SEQ ID NO: 87 is the sequence SFSANQEIRY SEVTPYHVT, which corresponds to positions 31-49 of SEQ ID NO: 1.

SEQ ID NO: 88 is the sequence DYQRNATEAY AKEVPHQIT, which corresponds to positions 36-54 of the IdeZ polypeptide NCBI Reference Sequence no WP_014622780.1.

SEQ ID NO: 89 is the sequence DDYQRNATEA YAKEVPHQIT, which may be present at the N terminus of a polypeptide of the invention.

### DETAILED DESCRIPTION OF THE INVENTION

Methods of Detecting Antibodies

The inventors have demonstrated in the examples that IgM antibodies can form immune complexes with IgG antibodies. These immune complexes can bind both target antigens and agents for detecting IgM, in the absence of any IgM antibodies that react with the target antigens. Such immune complexes therefore may generate false-positive results in assays for detecting IgM antibodies, in particular antigen-specific IgM antibodies. The examples also demonstrate that these problematic complexes can be cleaved with an IgG cysteine protease. IgA and IgE antibodies are known to form complexes with IgG in a similar manner to IgM. Therefore, the invention provides improved methods for detecting IgM, IgA and IgE antibodies that comprise contacting said sample with an IgG cysteine protease under conditions which permit IgG cysteine protease activity to occur.

The interference discovered by the inventors is unusual because it results in increased inappropriate signal, rather than a blocking of signal. The solution developed by the inventors using an IgG cysteine protease is particularly effective because it does not affect any IgM, IgA or IgE antibodies that are to be detected. Other techniques for addressing the interference identified by the inventors, such as depletion with anti-IgG antibodies or with columns, would likely result in a reduction in IgM, IgA and/or IgE levels.

The invention provides a method for detecting IgM antibodies, IgA antibodies or IgE antibodies in a sample comprising: contacting said sample with an IgG cysteine protease under conditions which permit IgG cysteine protease activity to occur; and contacting said sample with an agent suitable for detecting IgM antibodies, IgA antibodies or IgE antibodies.

Various immunoassays are available for detecting IgM, IgA and IgE antibodies and can be adapted to add a step of contacting a sample with an IgG cysteine protease.

In certain embodiments, the method of the invention is used in a solid phase assay such as ELISA and the agent for detecting the antibody is immobilised in the well of a plate. Contacting the sample with the IgG cysteine protease may take place in the same well.

In certain embodiments, the method of the invention is used in a solid phase assay such as a multiplex bead assay and the agent for detecting the antibody is immobilised on a bead. Different agents for detecting antibodies and/or different antigens of interest may be immobilised on different beads. In certain embodiments, the method of the invention is used in a single antigen bead assay. In certain embodiments the methods of the invention use the Luminex® platform.

In certain embodiments, the method of the invention is used in a solid phase assay, such as ELISA or a CAP FEIA (ImmunoCAP) test, and a target antigen of interest is immobilised on the solid substrate, such as the well or cellulose. The sample is then contacted with the immobilised antigen, contacted with an IgG cysteine protease, and contacted with an agent for detecting the antibody class of interest.

In certain embodiments, the method of the invention is used in a surface plasmon resonance assay and the agent for detecting the antibody is immobilised on a sensor chip surface.

In certain embodiments, the method of the invention is used in a nephelometry assay and the contacting of the sample with the agent suitable for detecting the antibodies is performed in solution.

In certain embodiments, the method of the invention is used in a bio-layer interferometry assay and the agent for detecting the antibody is immobilised on a biosensor tip.

In preferred embodiments, the agent suitable for detecting IgM antibodies, IgA antibodies or IgE antibodies is an anti-IgM antibody, an anti-IgA antibody or an anti-IgE antibody. Such antibodies are well known and widely available. In certain embodiments, the agent suitable for detecting IgM, IgA or IgE antibodies is labelled. Labelling can be used to aid detection.

In preferred embodiments, the method of the invention is for detecting IgM antibodies, IgA antibodies or IgE antibodies specific for a target of interest. As set out above, various immunoassays for detecting such antibodies are known and can be adapted to include a step of contacting the sample with an IgG cysteine protease. Generally, target-specific antibodies can be detected by contacting the sample with an antigen of interest under conditions that permit isolation of antibodies that bind specifically to the antigen of interest. In certain embodiments, the antigen of interest is fixed to a solid substrate or solid-phase matrix, such as a bead or a plate. The antigen may be coated on the solid substrate. In certain embodiments, the bead or plate is polystyrene. In certain embodiments, the bead is a or paramagnetic microsphere. In certain embodiments, the bead is labelled. Multiple beads may be labelled differently to enable differentiation of different beads and the detection of multiple different antibodies in a single assay. In certain embodiments, the method of the invention comprises sorting beads using flow cytometry.

It is within the skilled person's normal ability to determine appropriate cut-off values for any immunoassay utilising a step of cleavage with an IgG cysteine protease. Often, a cut-off>1000 MFI is used, and >3000 MFI was used in the examples.

In certain embodiments, the method of the invention utilises multiple antigens of interest and/or multiple agents for detecting IgM, IgA or IgE antibodies. Such multiplex assays allow antibodies against numerous different antigens to be detected in a single assay, and such assays will benefit from the improvement provided by use of an IgG cysteine protease. For example, in certain embodiments, the method comprises contacting the sample with multiple HLA class I and/or HLA class II antigens, such as at least 2, 3, 4, 5, 6, 7, 8, 9 or antigens. An exemplary assay may use beads with two alleles of each locus HLA-A, -B and -C; or HLA-DR, -DQ and -DP coating the bead, thus maintaining the ratio of expression similar to that observed on the cell surface. Alternatively, beads may be coated with multiple copies of a single antigen

The invention therefore provides a method for detecting IgM antibodies, IgA antibodies or IgE antibodies specific for an antigen of interest in a sample comprising: contacting said sample with an IgG cysteine protease under conditions which permit IgG cysteine protease activity to occur; contacting said sample with an antigen of interest, optionally fixed to a solid substrate, such as a plate or bead; and contacting said sample with an agent suitable for detecting IgM antibodies, IgA antibodies or IgE antibodies. Such methods will generally also involve standard washing steps. The step of contacting the sample with an IgG cysteine protease may occur before or after contacting the sample with an antigen of interest, but will generally occur before contacting the sample with an agent suitable for detecting IgM antibodies, IgA antibodies or IgE antibodies.

In certain embodiments, the method of the invention is practised in an ELISA format. In such an assay, the wells of an assay plate will typically be coated with an antibody target. An IgG protease is then added to the wells, followed by the sample to be tested for IgM, IgA or IgE antibodies that are specific for the target coating the wells. The protease and sample are allowed to interact under conditions suitable for IgG cysteine protease activity. After a suitable interval, the assay plate will be washed and a detector antibody which specifically binds to IgM, IgA or IgE antibodies will be added under conditions suitable for binding to the target-specific antibody. The detector antibody will bind to any intact target-specific antibody that has bound to the target in each well. After washing, the amount of detector antibody present in a well will be proportional to the amount of target-specific antibody bound to that well. The detector antibody may be conjugated directly or indirectly to a label or another reporter system (such as an enzyme), such that the amount of detector antibody remaining in each well can be determined.

Any suitable sample containing or suspected to contain IgM, IgA or IgE antibodies may be used in the methods of the invention. In preferred embodiments, the sample is a sample obtained from a patient, such as a serum sample.

Detecting Target-Specific Antibodies

In preferred embodiments, the methods of the invention detect the presence of IgM antibodies, IgA antibodies or IgE antibodies that are specific for a particular target or set of targets. As set out above, various immunoassays for detecting such antibodies are known and can be adapted to include a step of contacting the sample with an IgG cysteine protease. Generally, target-specific antibodies can be detected by contacting the sample with an antigen of interest under conditions that permit isolation of antibodies that bind specifically to the antigen of interest.

In preferred embodiments, the antigen of interest is a human antigen. Endogenous antibodies against such human antigens are often pathogenic, for example self-antigens can cause autoimmune diseases and donor-specific antigens can cause transplant rejection. In preferred embodiments the antigen of interest is a Human Leukocyte Antigen (HLA), an erythrocyte antigen, or is an antigen associated with an autoimmune disease, such as an antigen selected from Table A below. Detecting such antibodies is difficult and requires particular precision because clinicians often need to make accurate and nuanced assessments of patients' antibody signatures when diagnosing an autoimmune disease or determining whether a patient can receive a transplant from a particular donor. Therefore, the improvements achieved by the methods of the invention are particularly useful for detecting IgM antibodies, IgA antibodies or IgE antibodies specific for these HLA, erythrocyte antigens, or antigens associated with autoimmune diseases.

In further preferred embodiments, the methods of the invention are for use in detecting anti-drug antibodies. In such embodiments, the antigen of interest is a drug, such as an antibody or peptide drug, and IgM, IgA or IgE antibodies against the drug are detected. Such anti-drug antibodies can reduce the efficacy of a drug so their detection is useful for ensuring treatments can be adjusted as required to maintain efficacy. The methods of the invention are particularly useful for detecting anti-drug antibodies, because great precision is required to adjust a treatment regime appropriately.

Methods of Treating Diseases or Conditions Mediated by Pathogenic Antibodies and Immune Complexes

In preferred embodiments, the invention provides a method of treating in a subject a disease or condition mediated in whole or in part by pathogenic IgM antibodies, pathogenic IgA antibodies or pathogenic IgE antibodies, wherein the method comprises administering an IgG cysteine protease. The inventors have demonstrated in the examples that IgM antibodies can form immune complexes with IgG antibodies that can be cleaved with an IgG cysteine protease. IgA and IgE antibodies will likely form similar complexes with IgG antibodies. These immune complexes are expected to mediate disease processes, so IgG cysteine proteases will be useful for treating diseases or conditions mediated by pathogenic IgM antibodies, pathogenic IgA antibodies or pathogenic IgE antibodies.

The invention also provides an IgG cysteine protease, for use in a method of treating a disease or condition mediated in whole or in part by pathogenic IgM antibodies, pathogenic IgA antibodies or pathogenic IgE antibodies. The invention also provides use of an IgG cysteine protease in the manufacture of a medicament for use in a method of treating a disease or condition mediated in whole or in part by pathogenic IgM antibodies, pathogenic IgA antibodies or pathogenic IgE antibodies

In certain embodiments, the disease or condition mediated in whole or in part by pathogenic IgM antibodies, pathogenic IgA antibodies or pathogenic IgE antibodies is a disease selected from Table A.

In certain embodiments, the patient with a disease or condition mediated in whole or in part by pathogenic IgM antibodies, pathogenic IgA or pathogenic IgE antibodies that is to be treated according to the invention does not exhibit significant levels of anti-self IgG antibodies. In certain embodiments, the patient with a disease or condition mediated in whole or in part by pathogenic IgM antibodies, pathogenic IgA or pathogenic IgE antibodies that is to be treated according to the invention exhibits levels of anti-self IgG antibodies or other IgG antibodies that are sub-pathogenic or within normal ranges. The immune complexes identified by the inventors in the examples may mediate disease in the absence of anti-self IgG antibodies, and so IgG cysteine proteases will be useful for treating such diseases or conditions in the absence of significant levels of anti-self IgG antibodies.

In further preferred embodiments, the invention provides methods of treating in a subject a disease or condition mediated in whole or in part by pathogenic IgG antibodies, wherein said subject is determined to exhibit anti-IgG IgM antibodies, anti-IgG IgA antibodies or anti-IgG IgE antibodies, wherein the method comprises administering an IgG cysteine protease. The inventors have demonstrated in the examples that IgM antibodies can form immune complexes with IgG antibodies that can be cleaved with an IgG cysteine protease. IgA and IgE antibodies will likely form similar complexes with IgG antibodies. These immune complexes are expected to amplify the effects of pathogenic IgG antibodies, so an IgG cysteine protease will be especially effective at treating patients with anti-IgG IgM antibodies, anti-IgG IgA antibodies or anti-IgG IgE antibodies.

The invention also provides an IgG cysteine protease, for use in a method of treating in a subject a disease or condition mediated in whole or in part by pathogenic IgG antibodies, wherein said subject is determined to exhibit anti-IgG IgM antibodies, anti-IgG IgA antibodies or anti-IgG IgE antibodies. The invention also provides use of an IgG cysteine protease in the manufacture of a medicament for use in a method of treating in a subject a disease or condition mediated in whole or in part by pathogenic IgG antibodies, wherein said subject is determined to exhibit anti-IgG IgM antibodies, anti-IgG IgA antibodies or anti-IgG IgE antibodies.

In further preferred embodiments, the invention provides methods of treating in a subject a disease or condition mediated in whole or in part by complexes of IgG antibodies and IgM, IgA or IgE antibodies, wherein the method comprises administering an IgG cysteine protease. The inventors have identified in the examples that IgM and IgG antibodies can form complexes that can be cleaved by IgG cysteine proteases. IgA and IgE antibodies are expected to form similar complexes. Such immune complexes may be pathogenic and cause disease in, for example, the kidney or blood vessels. Thus, in preferred such embodiments, the invention provides a method for treating vasculitis, granulomatosis with polyangiitis (Wegener's granulomatosis), IgA nephropathy or systemic lupus erythematosus comprising administering an IgG cysteine protease. IgA nephropathy is caused by accumulation of IgA antibodies in the kidneys and this accumulation may be caused in part by complexes of IgA and IgG similar to those identified in the examples. Cleaving such complexes with IgG cysteine proteases as demonstrated in the examples may reduce or prevent accumulation of IgA in the kidneys, thereby preventing, treating or reducing symptoms of the disease. Vasculitis, granulomatosis with polyangiitis and systemic lupus erythematosus may be caused by autoantibodies and immune complexes that may be cleaved by IgG cysteine proteases in accordance with the invention. Complexes of IgG and IgE may cause or exacerbate acute allergic reactions or chronic inflammatory allergic diseases, and so in certain embodiments the invention provides an IgG cysteine protease, for use in a method of treating or preventing an acute allergic reaction or a chronic inflammatory allergic disease. In certain such embodiments, the subject to be treated may not exhibit significant levels of, or any, anti-self IgG antibodies or other pathogenic IgG antibodies. Instead, the complexes of IgG antibodies and IgM, IgA or IgE antibodies are themselves pathogenic.

The invention also provides an IgG cysteine protease, for use in a method of treating in a subject a disease or condition mediated in whole or in part by complexes of IgG antibodies and IgM, IgA or IgE antibodies. The invention also provides use of an IgG cysteine protease in the manufacture of a medicament for use in a method of treating in a subject a disease or condition mediated in whole or in part by complexes of IgG antibodies and IgM, IgA or IgE antibodies.

The pathogenic IgG, IgM, IgA or IgE antibodies forming immune complexes that are cleavable and treatable according to the invention may typically be specific for an antigen which is targeted in an autoimmune disease or other condition mediated wholly or in part by antibodies. Table A sets out a list of such diseases and the associated antigens. A protease of the invention may be used to treat any of these diseases or conditions. The protease is particularly effective for the treatment or prevention of autoimmune disease which is mediated in whole or in part by pathogenic IgG antibodies. Furthermore, the protease is especially effective for treatment of such diseases in patients with anti-IgG IgM antibodies, anti-IgG IgA antibodies or anti-IgG IgE antibodies. The protease is also particularly effective for the treatment or prevention of autoimmune disease which is mediated in whole or in part by pathogenic IgM antibodies, IgA antibodies or IgE antibodies.

TABLE A

| DISEASE | AUTOANTIGENS |
|---|---|
| ABO incompatible transplantation | ABO erythrocyte antigens |
| Addison’s disease | Steroid 21-hydroxylase, 17 alpha-Hydroxylase (17OH) and |
| | side-chain-cleavage enzyme (P450scc), Thyroperoxidase, |
| | thyroglobulin and H+/K(+)- |
| Anti-GBM glomerulonephritis | Anti-glomerular basement membrane (anti-GBM): |
| (related to Goodpasteur) | noncollagenous (NC1) domains of the alpha3alpha4alpha5(IV) |
| | collagen |
| Anti-neutrophil cytoplasmic | Myeloperoxidase, proteinase 3 |
| antibody-associated vasculitides | |
| (ANCA associated | |
| vasculitis)(Wegener granulomatosis, | |
| Churg-Strauss syndrome, microscopic | |
| polyangiitis) | |
| Anti-NMDAR Encephalitis | N-methyl-D-aspartate receptor (NMDAR) |
| Anti-phospholipid antibody | Negatively-charged phospholipids complexed with |
| syndrome (APS) and catastrophic | phospholipid binding plasma proteins (e.g. beta2GPI), |
| APS | cardiolipin, beta2-glycoprotein I, and (beta2GPI) |
| Autoimmune bullous skin diseases | IgG against keratinocytes. Specific target is desmoglein (Dsg) |
| (Pemphigus). Pemphigus foliaceus | 1 (desmosomal |
| (PF), fogo selvagem (FS)(endemic | Cadherins) |
| form), pemphigus vulgaris (PV) | |
| Autoimmune hemolytic anemia | Self-antigens on red-blood-cells |
| (AIHA) | |
| Autoimmune hepatitis (AIH) | Actin, antinuclear antibody (ANA), smooth muscle antibody |
| | (SMA), liver/kidney microsomal antibody (LKM-1), anti |
| | soluble liver antigen (SLA/LP) and anti-mitochondrial antibody |
| | (AMA), CYP2D6, CYP2C9-tienilic acid, UGT1A, CYP1A2, |
| | CYP2A6, CYP3A, CYP2E1, CYP11A1, CYP17 and CYP21 |
| Autoimmune neutropenia (AIN) | FcgRIIIb |
| Bullous pemphigoid (BP) | Hemidesmosomal proteins BP230 and BP180 (type XVII |
| | collagen), laminin 5, the alpha6 subunit of the integrin |
| | alpha6beta4 and p200 |
| Celiac disease | transglutaminase 2 (TG2), transglutaminase 3, actin, |
| | ganglioside, collagen, calreticulin and zonulin, thyroid, |
| | endocrine pancreas, anti-gastric and liver, anti-nuclear |
| | constituents, anti-reticulin, actin, smooth muscle, calreticulin, |
| | desmin, collagens, bone, anti-brain, ganglioside, neuronal, blood |
| | vessel |
| Chronic utricaria | Alpha-subunit of the high-affinity IgE receptor, IgE |
| Complete congenital heart block | Ro (Sjögens syndrome antigen A (SSA)), La (Sjögens syndrome |
| (CCHB) | antigen B(SSB)) |
| Diabetes type 1A (TIDM) | Islet cell autoantibodies (ICA), antibodies to insulin (IAA), |
| | glutamic acid decarboxylase (GAA or GAD), protein tyrosine |
| | phosphatase (IA2 or ICA512), Insulinoma Associated Peptide- |
| | 2. The number of antibodies, rather than the individual antibody, |
| | is thought to be most predictive of progression to overt diabetes. |
| Epidermolysis bullosa acquisita | The 145-kDa noncollagenous aminoterminal (NC-1) domain of |
| (EBA) | collagen VII |
| Essential mixed cryoglobulinemia | Essential mixed cryoglobulinemia antigens |
| Goodpasture's syndrome (also known | alpha3(IV) collagen (=Goodpasture antigen) |
| as Goodpasture’s disease and | |
| anti-glomerular basement membrane | |
| disease | |
| Graves'disease (Basedow's disease), | Thyrotropin receptor (TSHR) Thyroid peroxidase (TPO) |
| includes Goitre and hyperthyroidism, | |
| infiltrative exopthalmos and | |
| infiltarative dermopathy. | |
| Guillain-Barré syndrome (GBS). | Gangliosides GM1, GM1b, GD1a, and GalNAc-GD1a, |
| Acute inflammatory demyelinating | glycosphingolipid, myelin proteins PMP22 and P0 |
| polyneuropathy (AIDP), acute motor | |
| axonal neuropathy (AMAN) | |
| Hemophilia - Acquired FVIII | Factor VIII |
| deficiency | |
| IgA nephritis | |
| Idiopathic thrombocytopenic purpura | Platelet glycoprotein (GP) IIb-IIIa and/or GPIb-IX |
| (ITP) | |
| Lambert-Eaton myasthenic syndrome | voltage gated calcium channels |
| (LEMS) | |
| Mixed Connective Tissue Disease | IgG directed against the spliceosome, U1-snRNP |
| (MCTD) | |
| Multiple Myeloma | Multiple Myeloma antigens |
| Myasthenia gravis | Acetylcholine receptors (AchR), muscle-specific kinase |
| Myasthenic crisis | (MuSK) |
| Myocarditis, dilated cardiomyopathy | heart-reactive autoantibodies against multiple antigens e.g. |
| (DCM)(congestive cardiomyopathy) | cardiac myosin |
| Nemomyelitis Optica (NMO) | Aquaporin 4 (AQP4) |
| Primary biliary cirrhosis (PBC) | pyruvate dehydrogenase complex (PDC)-E2 and other members |
| | of the oxaloacid dehydrogenase family, Glycoprotein-210, p62, |
| | sp100 |
| Primary Progressive Multiple | Myelin oligodendrocyte glycoprotein (MOG), Myelin |
| Sclerosis (PPMS) | proteolipid protein (PLP), |
| | transketolase (TK), cyclic nucleotide phosphodiesterase type I |
| | (CNPase I), |
| | collapsin response mediator protein 2, tubulin beta4, |
| | neurofascin |
| Rheumatic heart disease (RHD), | Cardiac myosin |
| (Rheumatic fever) | |
| Rheumatoid Arthritis (RA) | Type II collagen, citrullin (-ated proteins (e.g. (fibrinogen, |
| | vimentin, filaggrin, type II collagen, enolase)), G6PI, RFs (anti- |
| | Fc/IgG), Vimentin, and cytokeratin |
| Serum-sickness, immune complex | Various antigens |
| hypersensitivity (type III) | |
| Sjögren Syndrome (SS) | Ro (Sjögens syndrome antigen A (SS-A)), La (Sjögens |
| | syndrome antigen B(SS-B)), p80 coilin, antinuclear antibodies, |
| | anti-thyroid, anti-centromere antibodies (Raynaud's |
| | phenomenon), anti-carbonic anhydrase II (distal renal |
| | tubular acidosis), anti-mitochondrial antibodies (liver |
| | pathology), cryoglobulins (evolution to non-Hodgkin's |
| | lymphoma). alpha- and beta-fodrin, islet cell autoantigen, |
| | poly(ADP)ribose polymerase (PARP), NuMA, Golgins, NOR- |
| | 90, M3-muscarinic receptor |
| SLE including Lupus nephritis | Autoantibodies to nuclear constituents (e.g. dsDNA and |
| | nucleosomes), dsDNA, PARP, Sm, PCDA, rRNA Ribosome P |
| | proteins, C1q |
| Stiff-person syndrome (SPS) | glutamic acid decarboxylase (GAD), amphiphysin. |
| Systemic sclerosis (scleroderma) | DNA-topoisomerase I (Scl-70), U3 snRNP, U2 snRNP, 7-2 |
| | RNP, NOR-90, centromere-associated proteins, and nucleolar |
| | antigens ,Anti-Th/To, Anti-RNA |
| | polymerase I/III, Anti-PDGF receptor, Anti-fibrillin-1, MS- |
| | muscarinic receptor, |
| Transplant rejection | Transplant rejection antigens |
| Thrombotic Thrombocytopenic | ADAMTS13 |
| Purpura (TTP) | |
| Wegener's granulomatosis | |
| (granulomatosis with polyangiitis) | |
| Anti-drug antibodies (ADA) | Any treatment which suffers from reduced efficacy due to the |
| | presence of antibodies specific for the therapeutic agent. |
| | Includes, for example, antibody-based therapeutics, gene |
| | therapy vectors, and the like. |

The IgG cysteine proteases in accordance with the present invention may also be used in therapy or prophylaxis. In therapeutic applications, proteases are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, proteases are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means.

In the methods of the invention, the protease may be co-administered with an immune-suppressive agent. In the methods of the invention, the protease is preferably administered by intravenous infusion, but may be administered by any suitable route including, for example, intradermal, subcutaneous, percutaneous, intramuscular, intra-arterial, intraperitoneal, intraarticular, intraosseous or other appropriate administration routes. The amount of the protease that is administered may be between 0.01 mg/kg BW and 2 mg/kg BW, between 0.05 and 1.5 mg/kg BW, between 0.1 mg/kg BW and 1 mg/kg BW, preferably between 0.15 mg/kg and 0.7 mg/kg BW and most preferably between 0.2 mg/kg and 0.3 mg/kg BW, in particular 0.25 mg/kg BW. The protease may be administered on multiple occasions to the same subject, provided that the quantity of anti-drug antibody (ADA) in the serum of the subject which is capable of binding to the protease does not exceed a threshold determined by the clinician. The quantity of ADA in the serum of the subject which is capable of binding to the protease may be determined by any suitable method, such as an agent specific CAP FEIA (ImmunoCAP) test or a titre assay.

### Organ Transplant

The therapeutic methods of the invention may be particularly useful in the context of organ transplants. The organ may be selected from kidney, liver, heart, pancreas, lung, or small intestine. The subject to be treated may preferably be sensitized or highly sensitized. By “sensitized” it is meant that the subject has developed antibodies to human major histocompatibility (MHC) antigens (also referred to as human leukocyte antigens (HLA)). The subject may have IgM, IgA or IgE anti-HLA antibodies, or the subject may have IgG anti-HLA antibodies and anti-IgG IgM, IgA or IgE antibodies. The anti-HLA antibodies originate from allogenically sensitized B-cells and are usually present in patients that have previously been sensitized by blood transfusion, previous transplantation or pregnancy (Jordan et al., 2003).

Whether or not a potential transplant recipient is sensitized may be determined by any suitable method. For example, a Panel Reactive Antibody (PRA) test may be used to determine if a recipient is sensitized. A PRA score >30% is typically taken to mean that the patient is “high immunologic risk” or “sensitized”. Alternatively, a cross match test may be conducted, in which a sample of the potential transplant donor's blood is mixed with that of the intended recipient. A positive cross-match means that the recipient has antibodies which react to the donor sample, indicating that the recipient is sensitized and transplantation should not occur. Cross-match tests are typically conducted as a final check immediately prior to transplantation.

IgG Cysteine Proteases

The inventors have demonstrated that use of an IgG cysteine protease in a method of detecting IgM, IgA or IgE antibodies leads to improved specificity and sensitivity because the protease is able to cleave immune complexes that can interfere with detection of IgM, IgA and IgE antibodies. The IgG cysteine protease for use with the invention is specific for IgG and does not have significant cleavage activity against the antibodies that are to be detected. Therefore, in certain embodiments, the protease cleaves IgG and does not cleave IgM. In certain embodiments, the protease cleaves IgG and does not cleave IgA. In certain embodiments, the protease cleaves IgG and does not cleave IgE. In certain embodiments, the protease cleaves IgG and does not cleave IgM, IgA or IgE.

In preferred embodiments, the protease for use in the methods of the invention is imlifidase (IdeS) (Immunoglobulin G-degrading enzyme of S. pyogenes). IdeS is an extracellular cysteine protease produced by the human pathogen S. pyogenes. IdeS was originally isolated from a group A Streptococcus strain of serotype M1, but the ides gene has now been identified in all tested group A Streptococcus strains. IdeS has an extraordinarily high degree of substrate specificity, with its only identified substrate being IgG. IdeS catalyses a single proteolytic cleavage in the lower hinge region of the heavy chains of all subclasses of human IgG. IdeS also catalyses an equivalent cleavage of the heavy chains of some subclasses of IgG in various animals. IdeS efficiently cleaves IgG to Fc and F(ab′)₂ fragments via a two-stage mechanism. In the first stage, one (first) heavy chain of IgG is cleaved to generate a single cleaved IgG (scIgG) molecule with a non-covalently bound Fc molecule. The scIgG molecule is effectively an intermediate product which retains the remaining (second) heavy chain of the original IgG molecule. In the second stage of the mechanism this second heavy chain is cleaved by IdeS to release a F(ab′)₂ fragment and a homodimeric Fc fragment. These are the products generally observed under physiological conditions. Under reducing conditions the F(ab′)₂ fragment may dissociate to two Fab fragments and the homodimeric Fc may dissociate into its component monomers. SEQ ID NO: 1 is the full sequence of IdeS including the N terminal methionine and signal sequence. It is also available as NCBI Reference sequence no. WP_010922160.1. SEQ ID NO: 2 is the mature sequence of IdeS, lacking the N terminal methionine and signal sequence. It is also available as Genbank accession no. ADF13949.1.

In alternative embodiments, the protease for use in the methods of the invention is IdeZ, which is a IgG cysteine protease produced by Streptococcus equi ssp. Zooepidemicus, a bacterium predominantly found in horses. SEQ ID NO: 3 is the full sequence of IdeZ including N terminal methionine and signal sequence. It is also available as NCBI Reference sequence no. WP_014622780.1. SEQ ID NO: 4 is the mature sequence of IdeZ, lacking the N terminal methionine and signal sequence.

In alternative embodiments, the protease for use in the methods of the invention is a hybrid IdeS/Z, such as that of SEQ ID NO: 5. The N terminus is based on IdeZ lacking the N terminal methionine and signal sequence.

In preferred embodiments, the protease for use in the invention may comprise or consist of SEQ ID NO: 2, 4 or 5. Proteases for use in the invention may comprise an additional methionine (M) residue at the N terminus and/or a tag at the C terminus to assist with expression in and isolation from standard bacterial expression systems. Suitable tags include a histidine tag which may be joined directly to the C terminus of a polypeptide or joined indirectly by any suitable linker sequence, such as 3, 4 or 5 glycine residues. The histidine tag typically consists of six histidine residues, although it can be longer than this, typically up to 7, 8, 9, 10 or 20 amino acids or shorter, for example 5, 4, 3, 2 or 1 amino acids.

In further preferred embodiments, the protease for use in the invention may comprise, consist essentially, or consist of the sequence of any one of SEQ ID NOs: 6 to 25. These sequences represent IdeS and IdeZ polypeptides with increased protease activity and/or reduced immunogenicity. Each of SEQ ID NOs: 6 to 25 may optionally include an additional methionine at the N terminus and/or a histidine tag at the C terminus. The histidine tag preferably consists of six histidine residues. The histidine tag is preferably linked to the C terminus by a linker of 3×glycine or 5×glycine residues.

In further preferred embodiments, the protease for use in the invention may comprise, consist essentially, or consist of the sequence of any one of SEQ ID NOs: 56 to 69. These sequences represent IdeS polypeptides with increased protease activity and/or reduced immunogenicity. Each of SEQ ID NOs: 56 to 69 may optionally include an additional methionine at the N terminus and/or a histidine tag at the C terminus. The histidine tag preferably consists of six histidine residues. The histidine tag is preferably linked to the C terminus by a linker of 3×glycine or 5×glycine residues.

In further preferred embodiments, the protease for use in the invention may comprise, consist essentially, or consist of the sequence of any one of SEQ ID NOs: 6 to 25, optionally with up to 3 (such as 1, 2 or 3) amino acid substitutions. Each of SEQ ID NOs: 6 to 25 and variants thereof may optionally include an additional methionine at the N terminus and/or a histidine tag at the C terminus.

In further preferred embodiments, the protease for use in the invention may comprise, consist essentially, or consist of the sequence of any one of SEQ ID NOs: 56 to 69, optionally with up to 3 (such as 1, 2 or 3) amino acid substitutions. Each of SEQ ID NOs: 56 to 69 and variants thereof may optionally include an additional methionine at the N terminus and/or a histidine tag at the C terminus.

The polypeptide of the invention is typically at least 100, 150, 200, 250, 260, 270, 280, 290, 300 or 310 amino acids in length. The polypeptide of the invention is typically no larger than 400, 350, 340, 330, 320 or 315 amino acids in length. It will be appreciated that any of the above listed lower limits may be combined with any of the above listed upper limits to provide a range for the length the polypeptide of the invention. For example, the polypeptide may be 100 to 400 amino acids in length, or 250 to 350 amino acids in length. The polypeptide is preferably 290 to 320 amino acids in length, most preferably 300 to 315 amino acids in length.

The primary structure (amino acid sequence) of a protease of the invention is based on the primary structure of IdeS, IdeZ or IdeS/Z, specifically the amino acid sequence of SEQ ID NO: 2, 4 or 5, respectively. The sequence of a protease of the invention may comprise a variant of the amino acid sequence of SEQ ID NO: 2, 4 or 5, which is at least 80% identical to the amino acid sequence of SEQ ID NO: 2, 4 or 5. The variant sequence may be at least 80%, at least, 85%, preferably at least 90%, at least 95%, at least 98% or at least 99% identical to the sequence of SEQ ID NO: 2, 4 or 5. The variant may be identical to the sequence of SEQ ID NO: 2, 4 or 5 apart from the inclusion of one or more of the specific modifications identified in WO2016/128558 or WO2016/128559. Identity relative to the sequence of SEQ ID NO: 2, 4 or 5 can be measured over a region of at least 50, at least 100, at least 200, at least 300 or more contiguous amino acids of the sequence shown in SEQ ID NO: 2, 4 or 5, or more preferably over the full length of SEQ ID NO: 4 or 5.

The protease for use in the invention may be an IdeS, IdeZ or IdeS/Z polypeptide that comprises a variant of the amino acid sequence of SEQ ID NO:, 2 4 or 5 in which modifications, such as amino acid additions, deletions or substitutions are made relative to the sequence of SEQ ID NO: 2, 4 or 5. Such modifications are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art.

IgG cysteine protease activity may be assessed by any suitable method, for example by incubating a polypeptide with a sample containing IgG and determining the presence of IgG cleavage products. Suitable methods are described in the WO2016/128559. Suitable assays include an ELISA-based assay, such as that which is described in WO2016/128559. In such an assay, the wells of an assay plate will typically be coated with an antibody target, such as bovine serum albumin (BSA). Samples of the polypeptide to be tested are then added to the wells, followed by samples of target-specific antibody that is antibody specific for BSA in this example. The polypeptide and antibody are allowed to interact under conditions suitable for IgG cysteine protease activity. After a suitable interval, the assay plate will be washed and a detector antibody which specifically binds to the target-specific antibody will be added under conditions suitable for binding to the target-specific antibody. The detector antibody will bind to any intact target-specific antibody that has bound to the target in each well. After washing, the amount of detector antibody present in a well will be proportional to the amount of target-specific antibody bound to that well. The detector antibody may be conjugated directly or indirectly to a label or another reporter system (such as an enzyme), such that the amount of detector antibody remaining in each well can be determined. The higher the potency of the tested polypeptide that was in a well, the less intact target-specific antibody will remain and thus there will be less detector antibody. Typically, at least one well on a given assay plate will include IdeS instead of a polypeptide to be tested, so that the potency of the tested polypeptides may be directly compared to the potency of IdeS. IdeZ and IdeS/Z may also be included for comparison.

Other assays may determine the potency of a tested polypeptide by directly visualizing and/or quantifying the fragments of IgG which result from cleavage of IgG by a tested polypeptide. An assay of this type is also described in WO2016/128559. Such an assay will typically incubate a sample of IgG with a test polypeptide (or with one or more of IdeS, IdeZ and IdeS/Z as a control) at differing concentrations in a titration series. The products which result from incubation at each concentration are then separated using gel electrophoresis, for example by SDS-PAGE. Whole IgG and the fragments which result from cleavage of IgG can then be identified by size and quantified by the intensity of staining with a suitable dye. The greater the quantity of cleavage fragments, the greater the potency of a tested polypeptide at a given concentration. A polypeptide of the invention will typically produce detectable quantities of cleavage fragments at a lower concentration (a lower point in the titration series) than IdeZ and/or IdeS. This type of assay may also enable the identification of test polypeptides that are more effective at cleaving the first or the second heavy chain of an IgG molecule, as the quantities of the different fragments resulting from each cleavage event may also be determined. A polypeptide of the invention may be more effective at cleaving the first chain of an IgG molecule than the second, particularly when the IgG is an IgG2 isotype. A polypeptide of the invention may be more effective at cleaving IgG1 than IgG2.

### Production of Polypeptides

A polypeptide as disclosed herein may be produced by any suitable means. For example, the polypeptide may be synthesised directly using standard techniques known in the art, such as Fmoc solid phase chemistry, Boc solid phase chemistry or by solution phase peptide synthesis. Alternatively, a polypeptide may be produced by transforming a cell, typically a bacterial cell, with a nucleic acid molecule or vector which encodes said polypeptide. Production of polypeptides by expression in bacterial host cells is described and exemplified in WO2016/128559.

### Compositions and Formulations Comprising Polypeptides

The present invention also provides compositions comprising a protease, for use in the therapeutic methods of the invention. For example, the invention provides a composition comprising one or more polypeptides of the invention, and at least one pharmaceutically acceptable carrier or diluent. The carrier (s) must be ‘acceptable’ in the sense of being compatible with the other ingredients of the composition and not deleterious to a subject to which the composition is administered. Typically, carriers and the final composition, are sterile and pyrogen free.

Formulation of a suitable composition can be carried out using standard pharmaceutical formulation chemistries and methodologies all of which are readily available to the reasonably skilled artisan. For example, the agent can be combined with one or more pharmaceutically acceptable excipients or vehicles. Auxiliary substances, such as wetting or emulsifying agents, pH buffering substances, reducing agents and the like, may be present in the excipient or vehicle. Suitable reducing agents include cysteine, thioglycerol, thioreducin, glutathione and the like. Excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e. g., sterile pyrogen-free water) prior to parenteral administration of the reconstituted composition. The compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. The compositions may be suitable for administration by any suitable route including, for example, intradermal, subcutaneous, percutaneous, intramuscular, intra-arterial, intraperitoneal, intraarticular, intraosseous or other appropriate administration routes. Preferred compositions are suitable for administration by intravenous infusion.

Kits

The invention also provides kits for practising the methods of the invention. The kits of the invention an IgG cysteine protease, as defined and discussed above, and an agent for detecting an IgM antibody, an IgA antibody or an IgE antibody, as defined and discussed above. In certain embodiments, the kit also comprises an antigen of interest, such as a Human Leukocyte Antigen (HLA), an erythrocyte antigen or a drug antigen. As discussed above, the antigen of interest may be fixed, such as coated, on a solid substrate, such as a plate or a bead.

### General

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes “polypeptides”, and the like.

Unless specifically prohibited, the steps of a method disclosed herein may be performed in any appropriate order and the order in which the steps are listed should not be considered limiting.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

The terms “patient” and “subject” are used interchangeably and typically refer to a human. References to IgG typically refer to human IgG unless otherwise stated.

Amino acid identity as discussed above may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et al (1984) Nucleic Acids Research 12, 387-395).

All publications, patents and patent applications cited herein, whether supra or infra, are hereby incorporated by reference in their entirety.

### Example 1

Unless indicated otherwise, the methods used are standard biochemistry and molecular biology techniques. Examples of suitable methodology textbooks include Sambrook et al., Molecular Cloning, A Laboratory Manual (1989) and Ausubel et al., Current Protocols in Molecular Biology (1995), John Wiley and Sons, Inc.

### Introduction

The immunoglobulin G-degrading cysteine protease, imlifidase (IdeS) is an IgG endopeptidase that is currently under development as a rapid desensitization treatment in kidney transplantation. Imlifidase is highly specific and cleaves all subclasses of human IgG. Due to its specific protease activity, imlifidase effectively prevents Fc-mediated effector functions of IgG, including antibody-dependent cellular phagocytosis (ADCP), antibody-dependent cytotoxicity (ADCC) and complement-dependent cytotoxicity (CDC).

The purpose of the present study was to further assess whether imlifidase has an effect on IgM antibodies (Abs), in particular in sensitized patients with end stage renal disease (ESRD), and to identify new uses for imlifidase.

### Materials and Methods

Serum samples from sensitised patients with end stage renal disease (ESRD) Serum samples from sensitised patients with end stage renal disease (ESRD) enrolled in a clinical phase II trial (13-HMedIdeS-02; NCT02224820) treated with imlifidase at 0.12 or 0.25 mg/kg were investigated. Study samples pre-dose and 24 hours post-dose imlifidase were studied.

Treatment with Imlifidase

Imlifidase (P16-0041701) was used for in vitro treatments.

SDS-PAGE and Immunoblotting

Imlifidase cleaved purified human IgM (1 mg/mL) (#16-16-090713-M, Athens Research & Technology) or human IgG and IgM in serum samples from sensitised patients with ESRD were separated under nonreducing conditions on 4-20% Mini-PROTEAN TGX Stain-Free gels (Bio-Rad) in Tris-Glycine-SDS buffer. The gels were transferred to a 0.45 μm nitrocellulose membranes and blocked in 5% nonfat milk (NFM) prior to incubation with PE-conjugated donkey anti-human IgM (#IGM-PEC1, One Lambda) in PBS-Tween.
Membranes were washed and analysed in blot settings suitable for PE. Signals were acquired using a ChemiDoc MP system (Bio-Rad).

IgG Purification Using CaptureSelect Affinity Matrix

CaptureSelect™ IgG-CH1 Affinity Matrix (#194320005, ThermoFisher Scientfic) was used for purification of serum samples. The CaptureSelect™ Affinity Matrix purifies recombinant human Fab fragments and IgG from complex source materials in a single step. The affinity matrix recognizes all four subclasses of IgG (IgG1, IgG2, IgG3 and IgG4), independent of the light chain type (kappa/lambda). Serum samples were analysed on SDS-PAGE for evaluation and purification of IgG.

Single Antigen Bead HLA Assay for Class I and II (One Lambda/ThermoFisher)

Serum samples from sensitised patients with ESRD treated with imlifidase at 0.12 or 0.25 mg/kg were investigated. Study samples pre-dose and 24 hours post-dose imlifidase were studied. All serum samples were pre-treated with EDTA to overcome the prozone effect. Sera were tested for both HLA class I and class II anti-HLA antibody using commercial Single Antigen Beads on the Luminex platform (LABScreen Single Antigen, #LS1A04, #LS2A01, One Lambda). The serum was first incubated with LABScreen beads for 30 minutes, washed for three times with wash buffer. Phycoeryrtin (PE)-conjugated goat anti-human IgG or PE-conjugated donkey anti-human IgM was added and incubated for 30 minutes, washed two times. The LABScan 100 analyzer was used to detect the fluorescent emission of PE from each bead. The reaction pattern of the test serum was compared to the lot-specific worksheet defining the antigen array and assigned the HLA specificity. The results were interpreted of using HLA Fusion software (One Lambda) and expressed as mean fluorescence intensity (MFI).

Rheumatoid Factor (RF)

Serum samples from sensitised patients with ESRD were screened for rheumatoid factor (IgM-RF) (Labmedicin Skåne, Clinical Immunology and Transfusion Medicine, Lund, Sweden).

Immunoglobin (Ig) Class and IgG Subclasses

Serum samples from two healthy volunteers were measured for immunoglobulin class and IgG subclasses pre-purification with CaptureSelect Matrix and post-purification (Labmedicin Skåne, Clinical Immunology and Transfusion Medicine, Lund, Sweden).

### Results

No Cleavage of Purified Human IgM was Seen after Incubation with Imlifidase

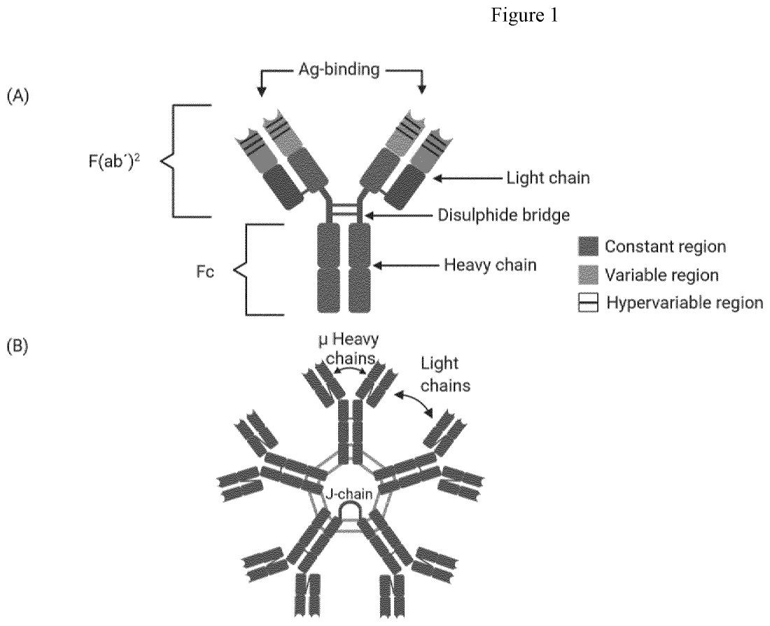

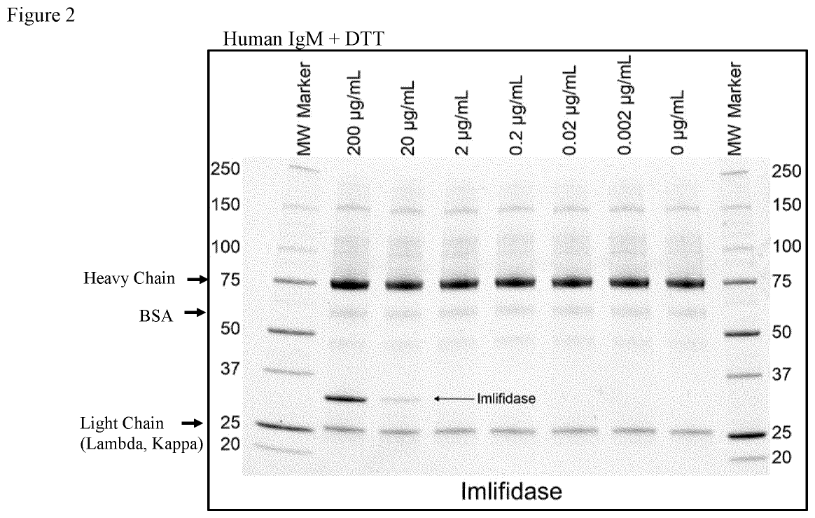

The capacity of imlifidase to cleave purified human IgM was evaluated by separating the test item on SDS-PAGE gel. DTT was added to digest IgM prior to heating, to destroy the disulphide bridges (see FIG. 1). No digestion of purified human IgM was seen after incubation with imlifidase in high concentrations (up to 200 μg/mL) for 2 hours at 37° C. (see FIG. 2).

Purification of IgG was Successfully Performed in Serum Using CaptureSelect Affinity Matrix

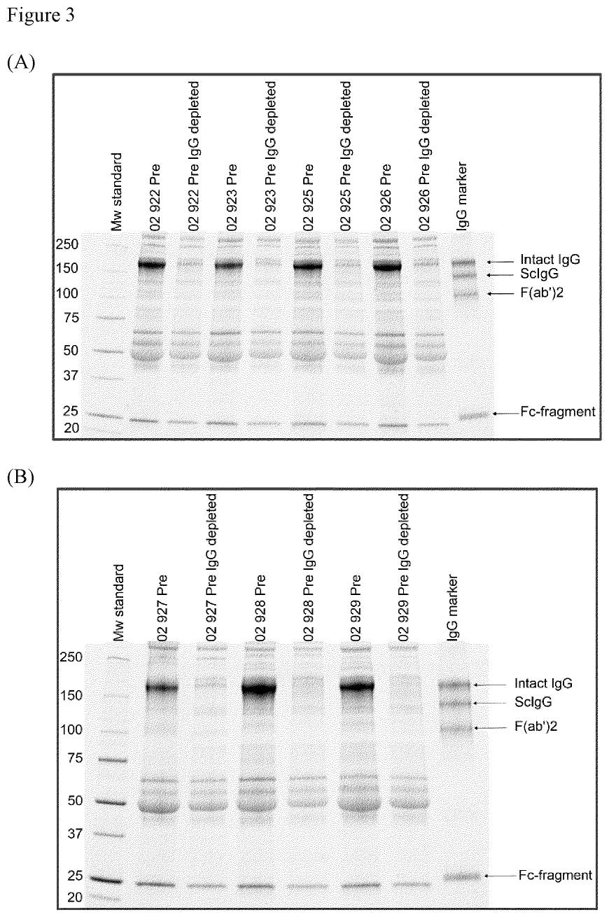

Serum samples were successfully purified using CaptureSelect IgG-CH1 Affinity Matrix and no intact IgG band could be detected on the SDS-PAGE gels compared to untreated serum samples (see FIG. 3).

Treatment with Imlifidase has Great Impact on the Level of Circulating Anti-HLA Antibodies of IgG Specificity

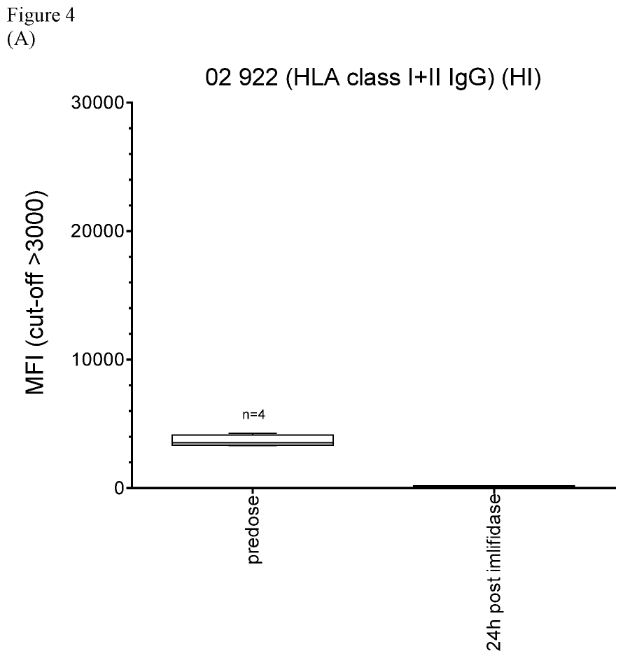

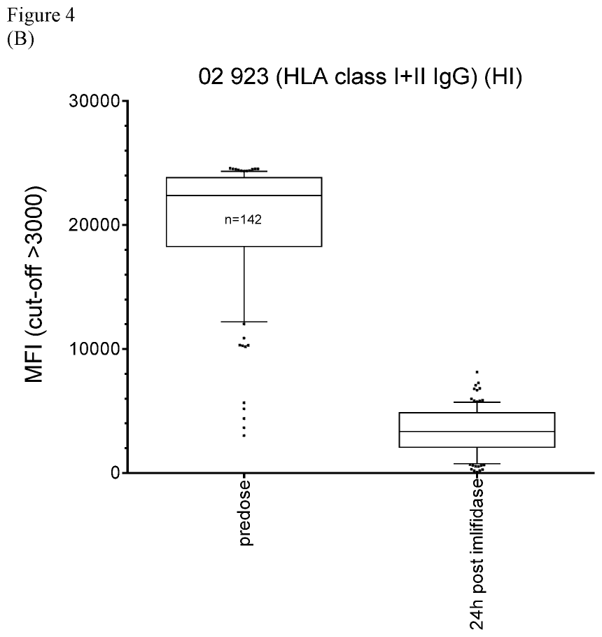

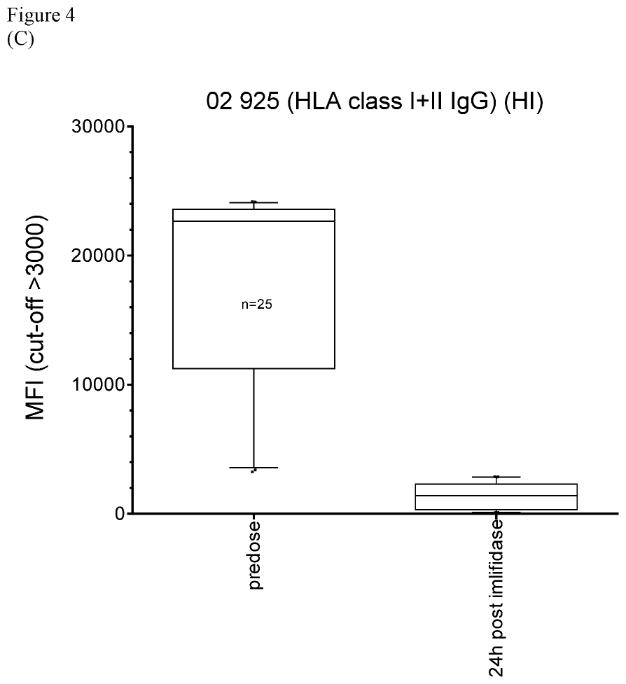

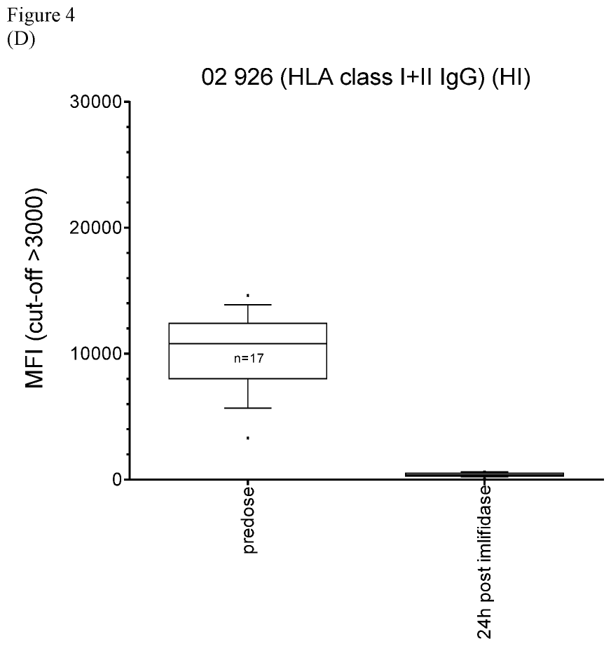

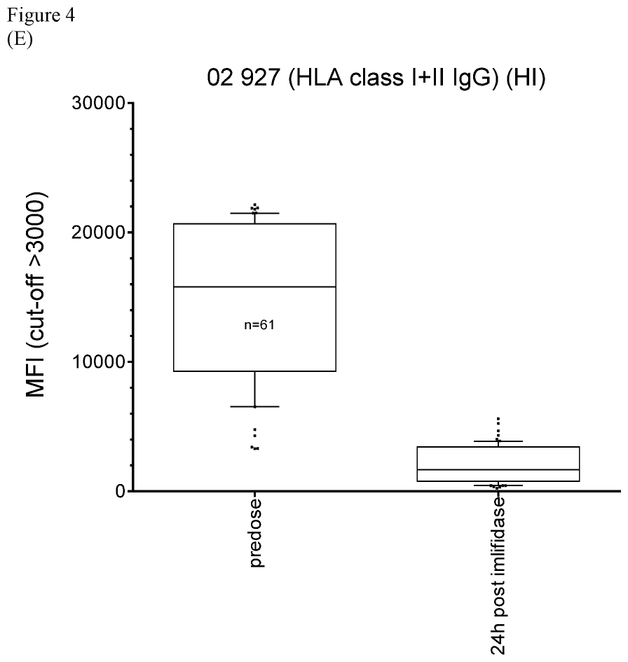

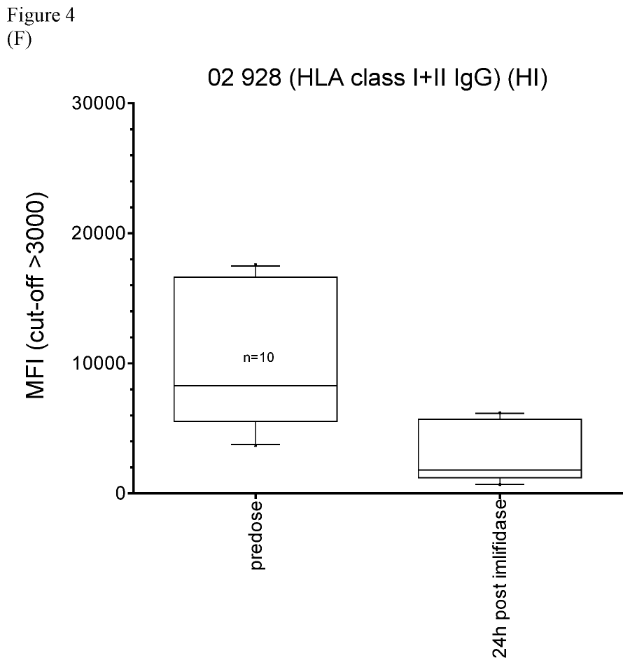

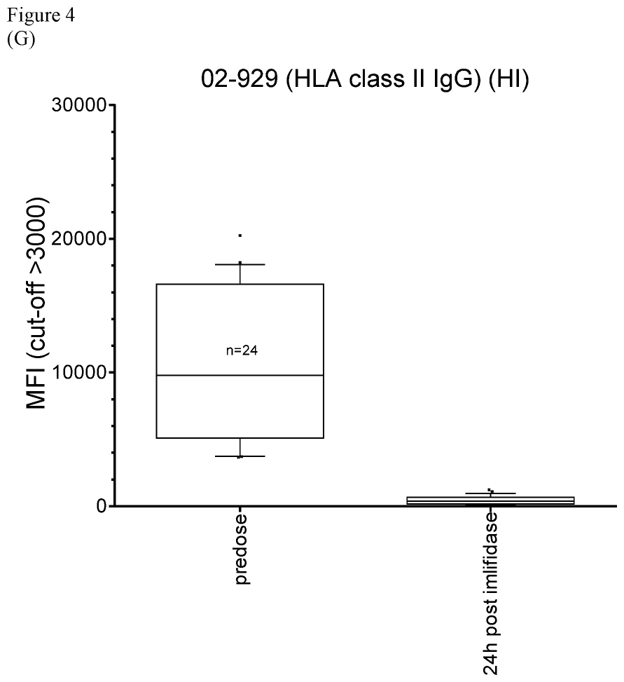

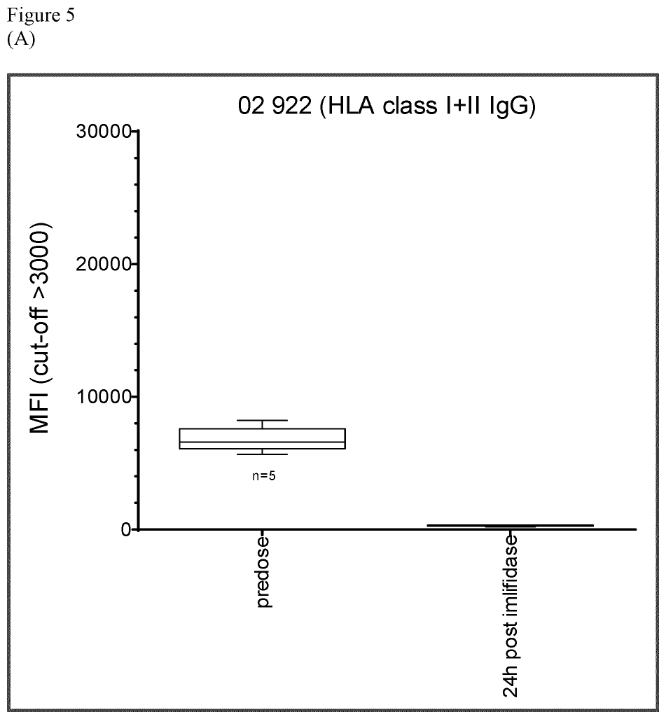

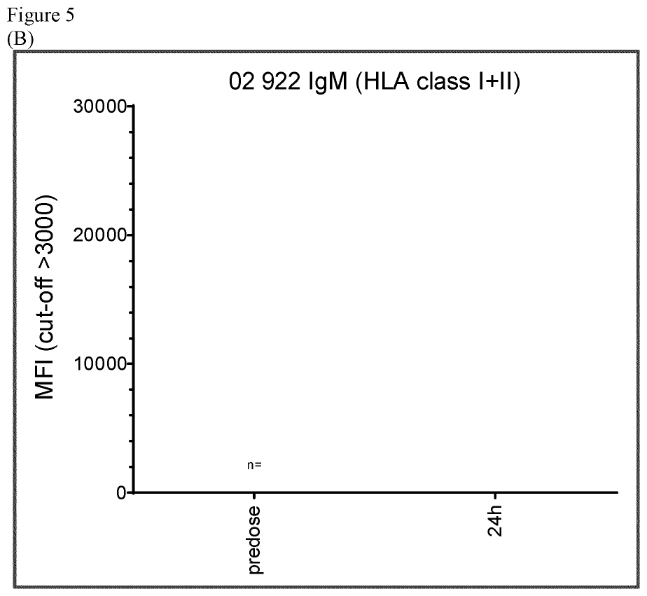

The ability of imlifidase to cleave IgG and thereby Donor Specific Antibodies (DSAs) and enable patients with a positive crossmatch with a deceased donor to undergo transplantation without requiring weeks of treatments pre-transplantation has been published earlier (Jordan et al., 2017; Lonze et al., 2018; Lorant et al., 2018).
Pre-dose and 24 hours post-imlifidase serum samples from sensitised patients with ESRD were studied. The cleavage of anti-HLA IgG antibodies by imlifidase could be detected in this study as well. A clear decrease of the level of circulating anti-HLA IgG antibodies of both class I and class II was seen in all seven patients (see FIG. 4).

Treatment with Imlifidase has No Impact on the Level of Circulating Anti-HLA Antibodies of IgM Specificity

An alleged ability of imlifidase to cleave IgM and thereby Donor Specific Antibodies (DSAs) with IgM specificity was presented at the ATC meeting 2019 (X Zhang, S. Jordan, 2019. Anti-HLA IgM Antibodies Are Reduced in Highly-HLA Sensitized Patients Transplanted after Imlifidase (IdeS) Treatment).

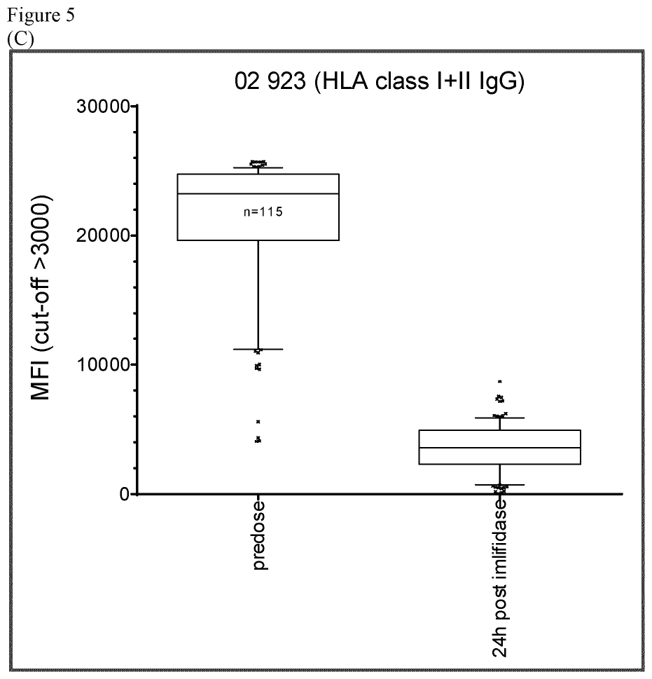

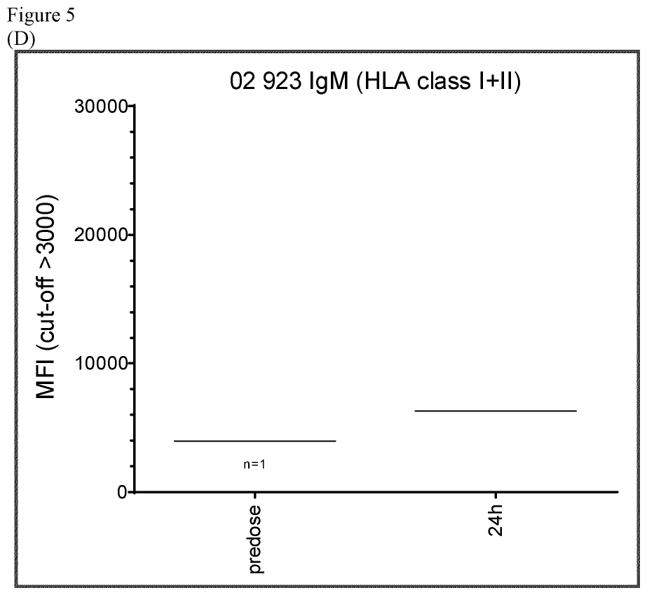

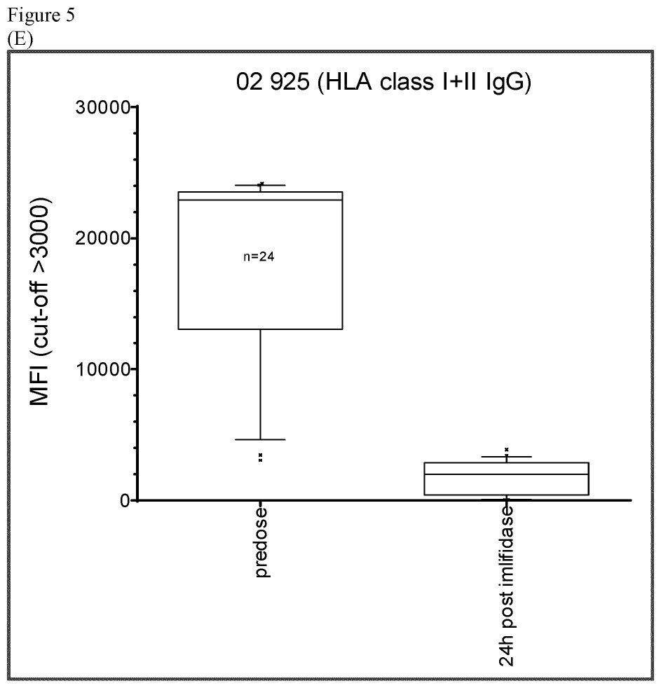

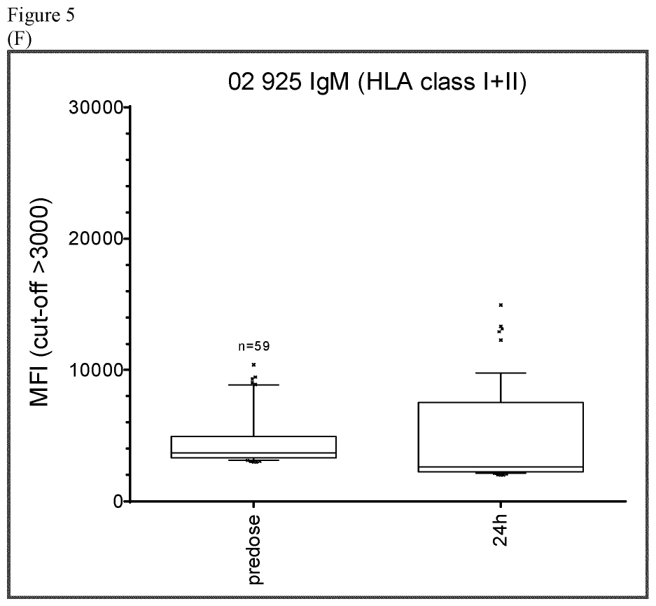

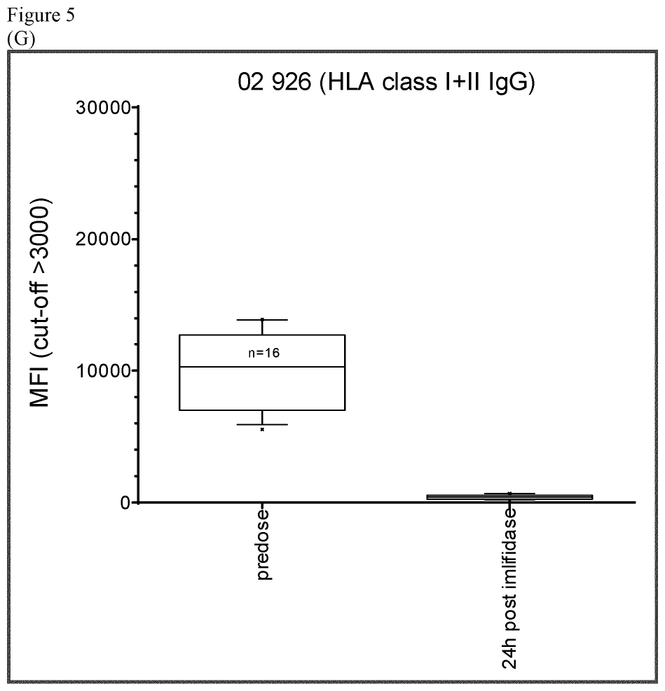

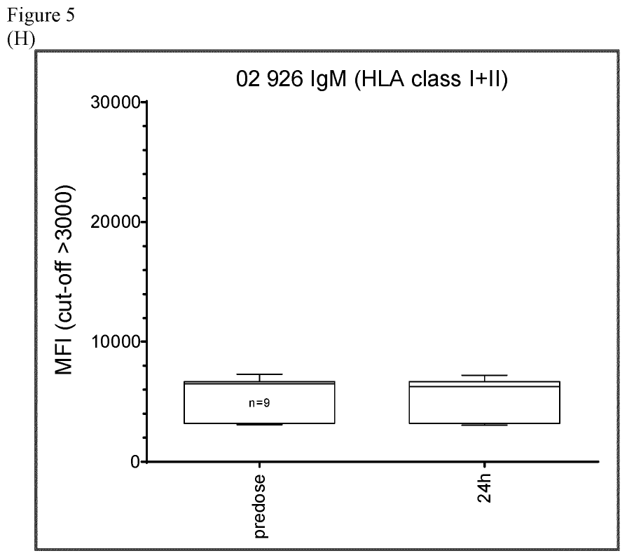

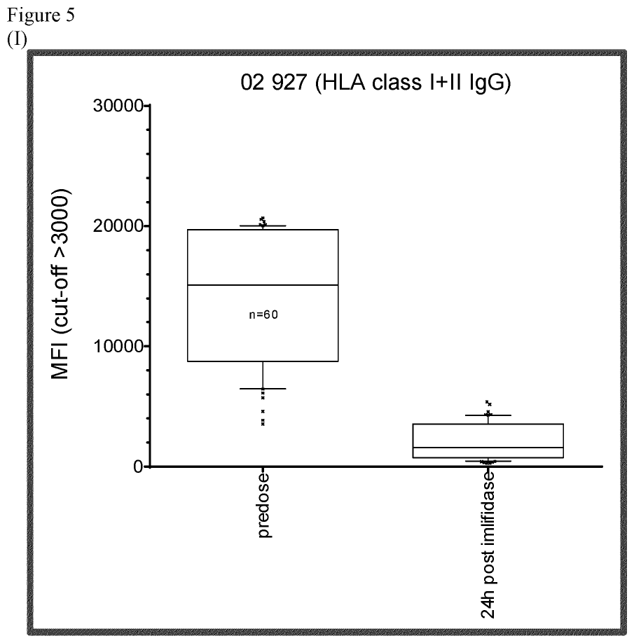

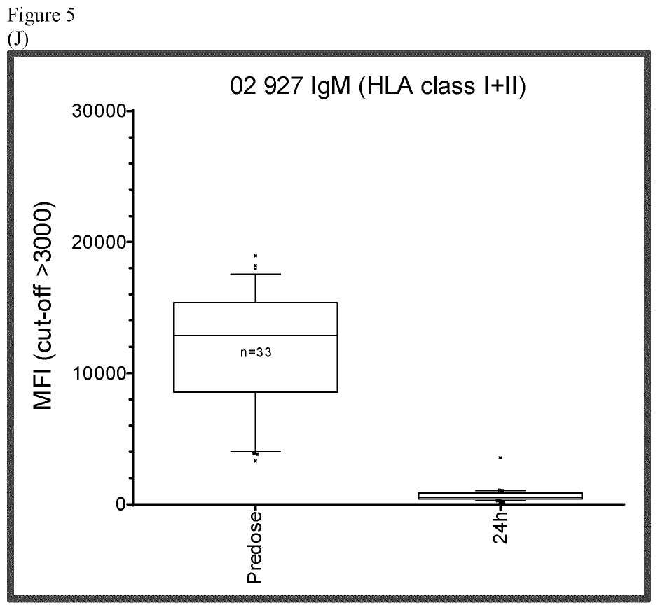

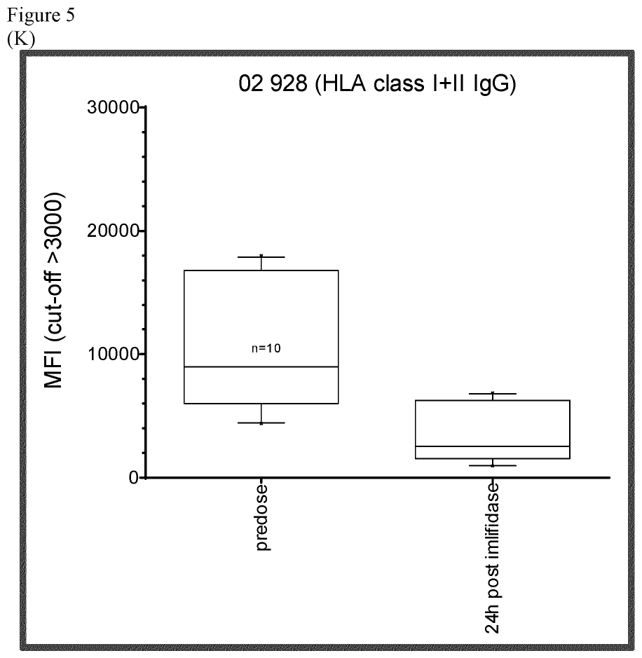

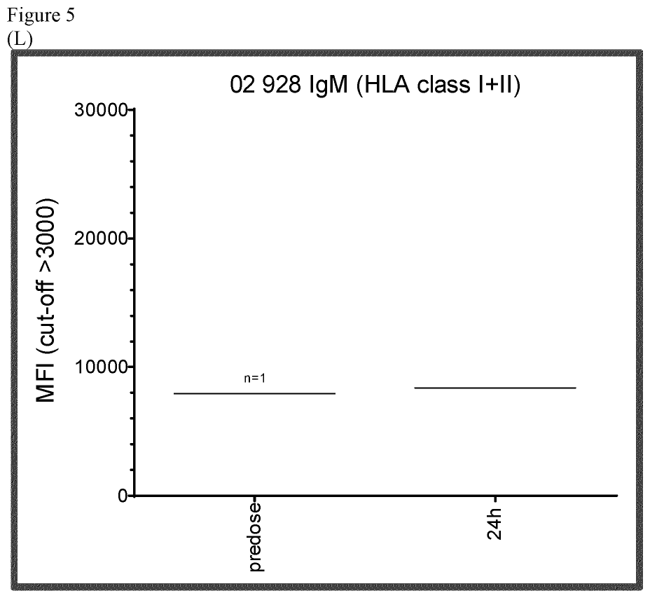

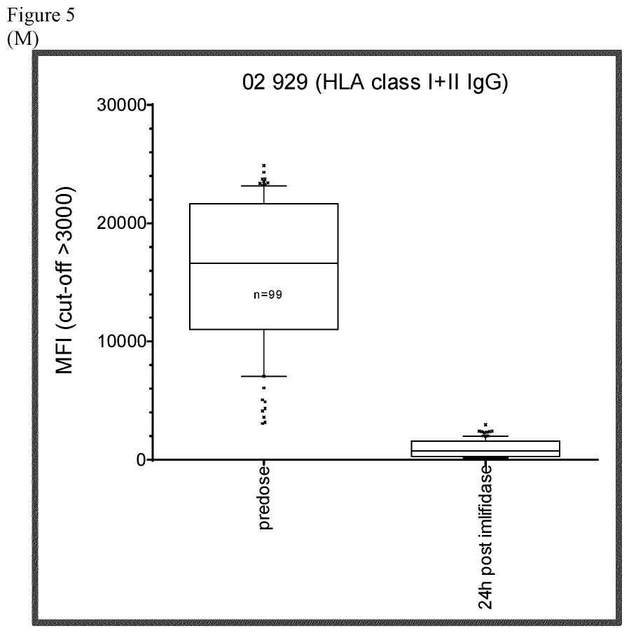

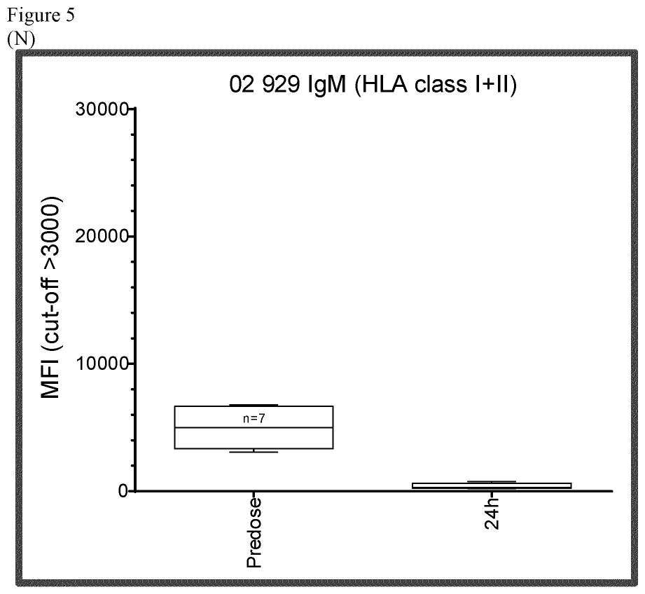

Pre-dose and 24 hours post imlifidase serum samples from sensitised patients with ESRD treated with imlifidase were studied. A reduction of circulating anti-HLA IgM antibodies after treatment with imlifidase was detected in 2 (subject 02-927 and subject 02-929) out of 7 patients (see FIG. 5).

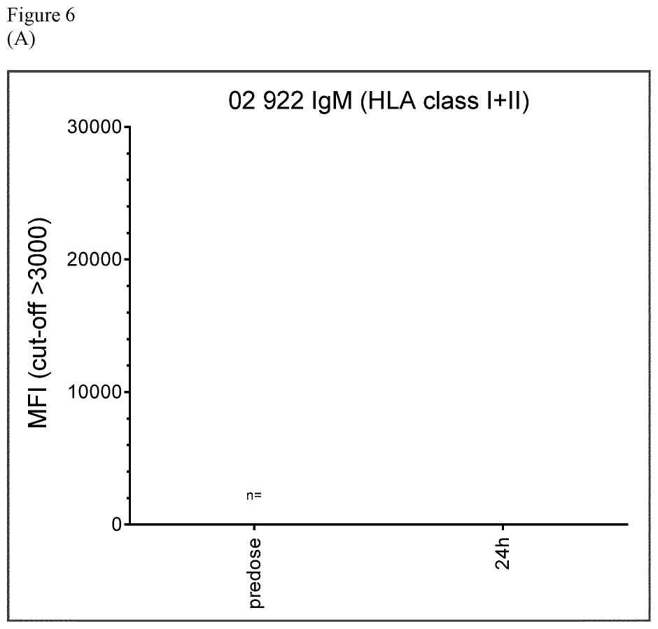

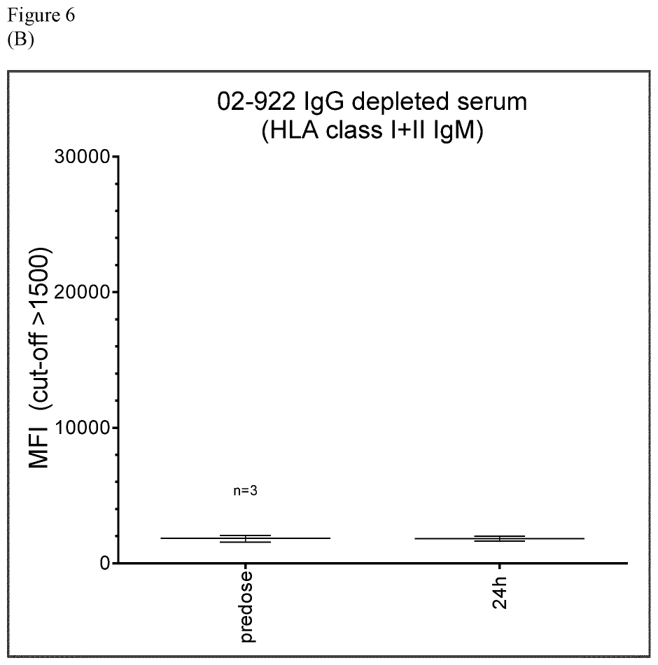

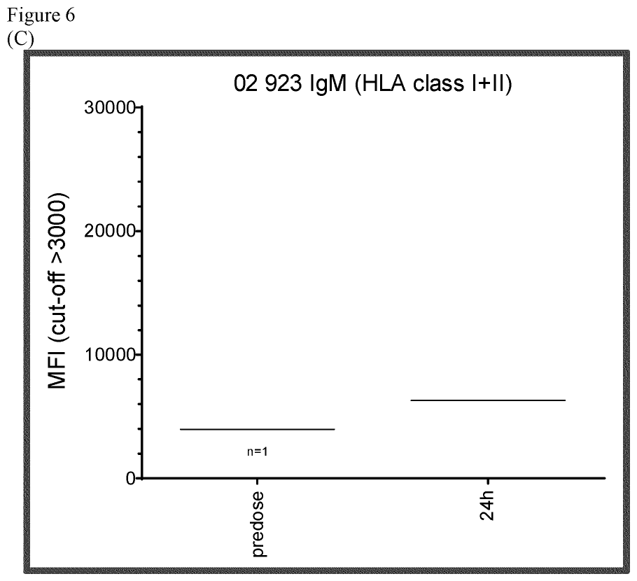

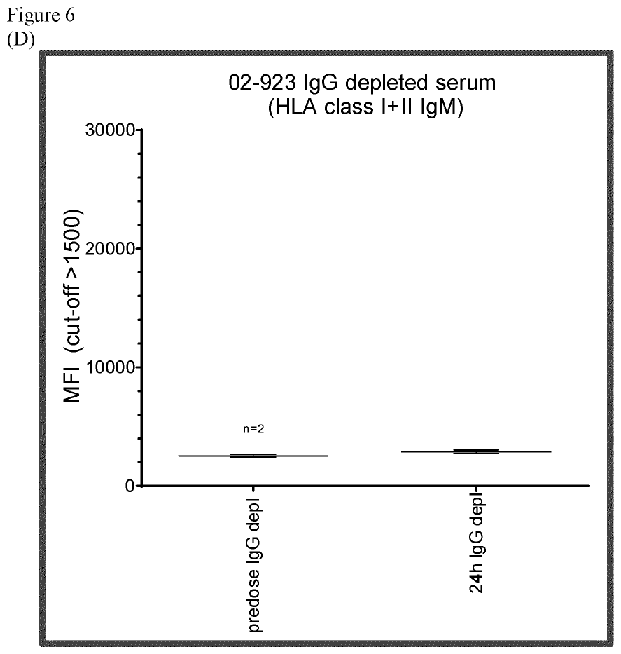

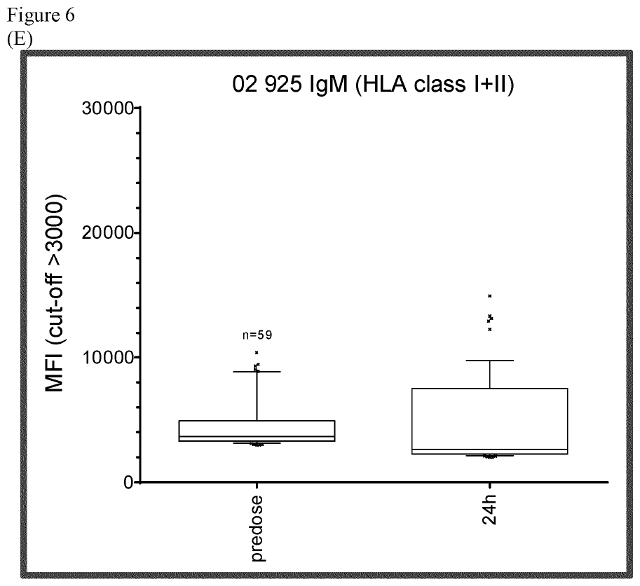

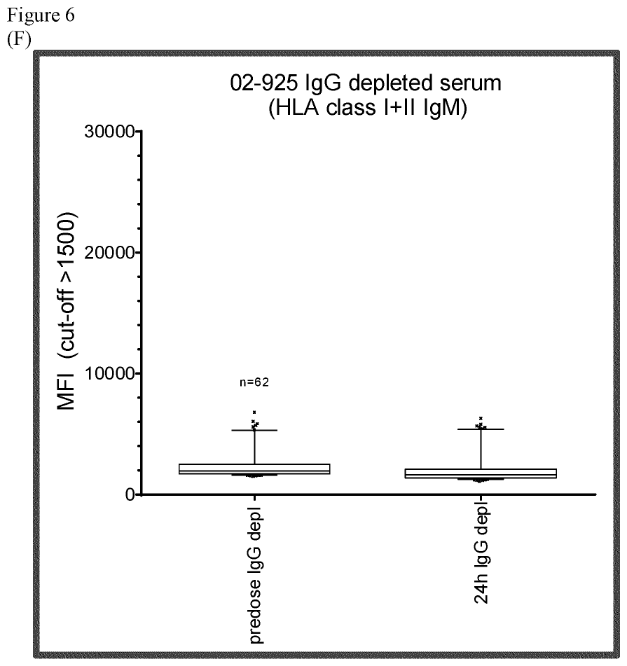

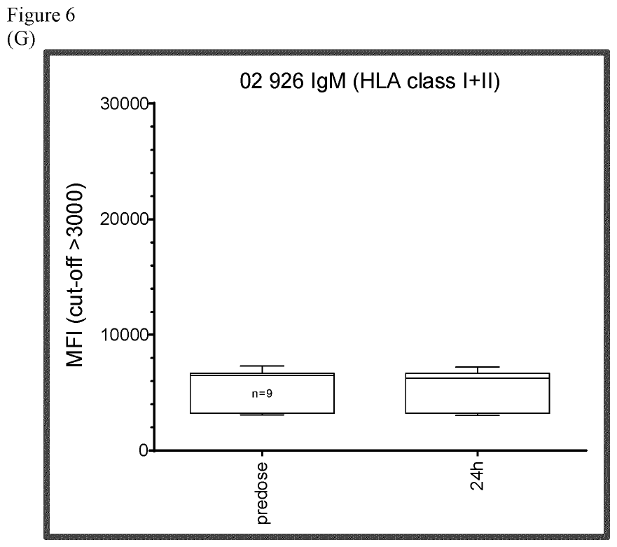

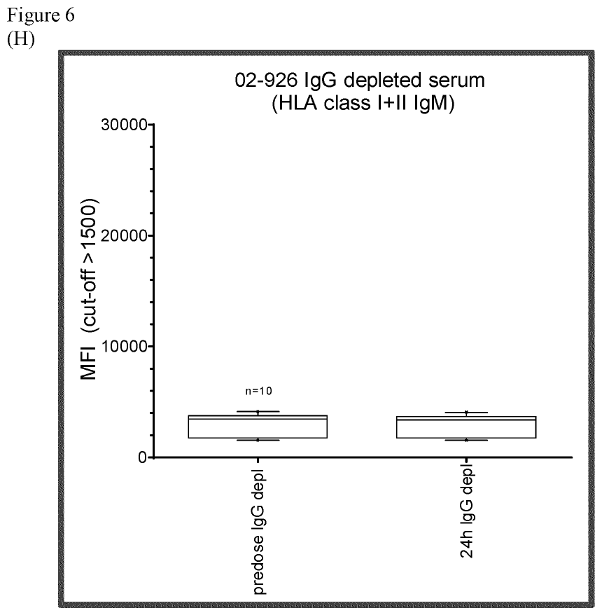

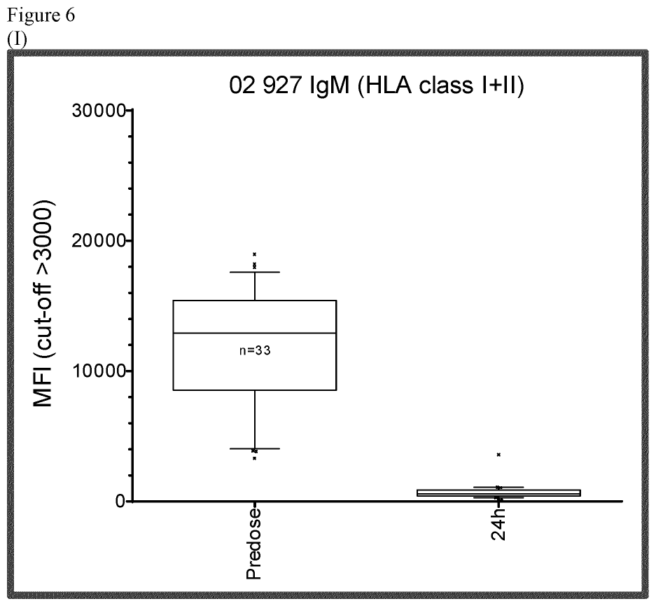

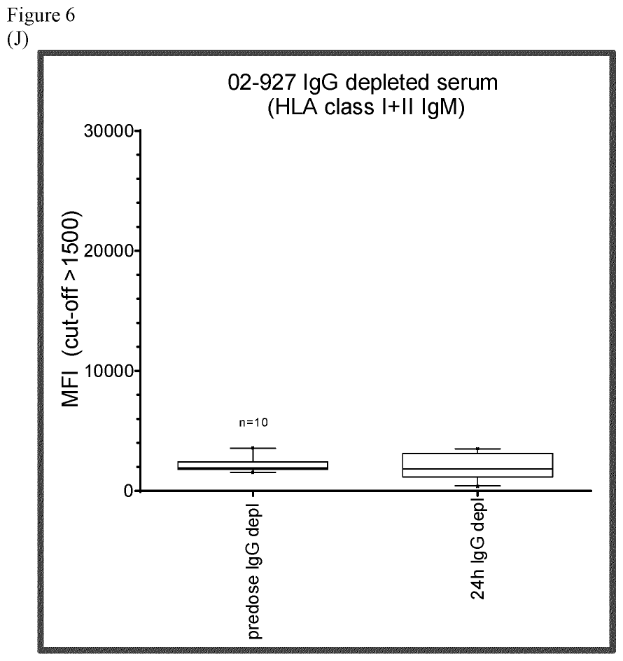

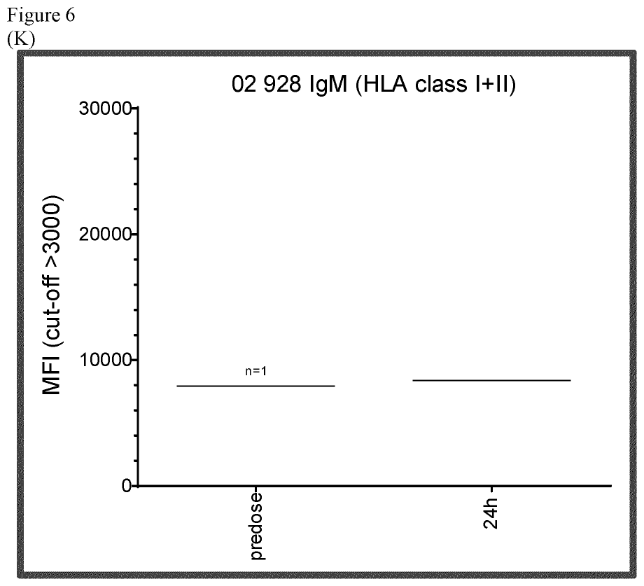

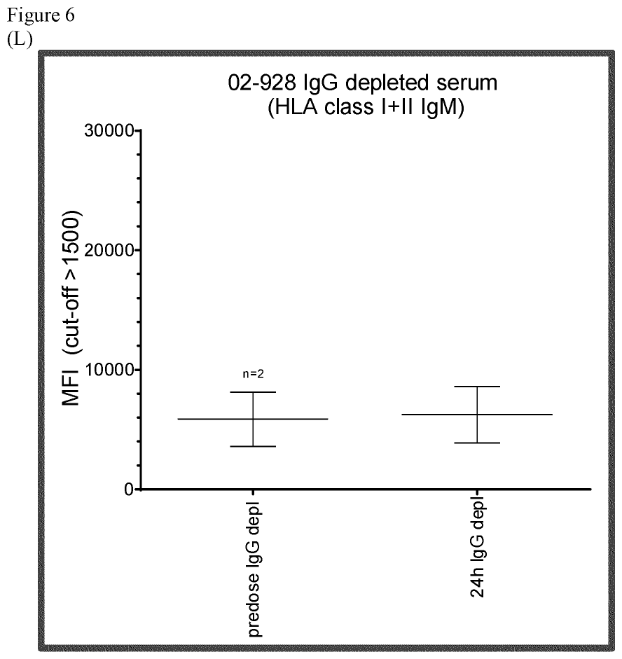

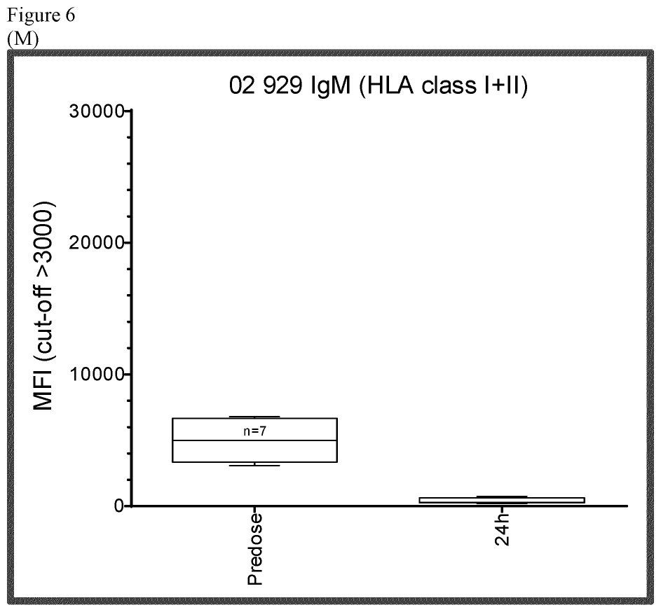

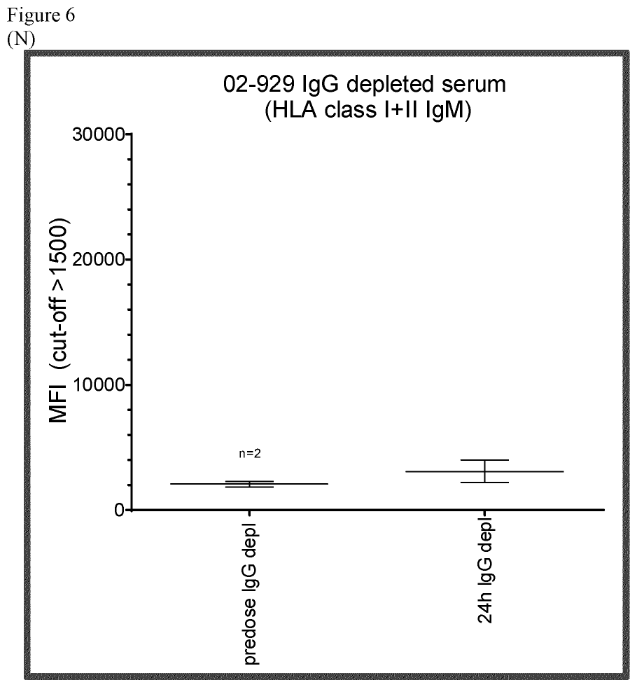

However, when serum samples were purified (IgG depleted) with CaptureSelect Matrix, no effect on the level of circulating anti-HLA IgM antibodies was seen after treatment with imlifidase (see FIG. 6).

Human IgM Cleavage by Imlifidase was not Detectable

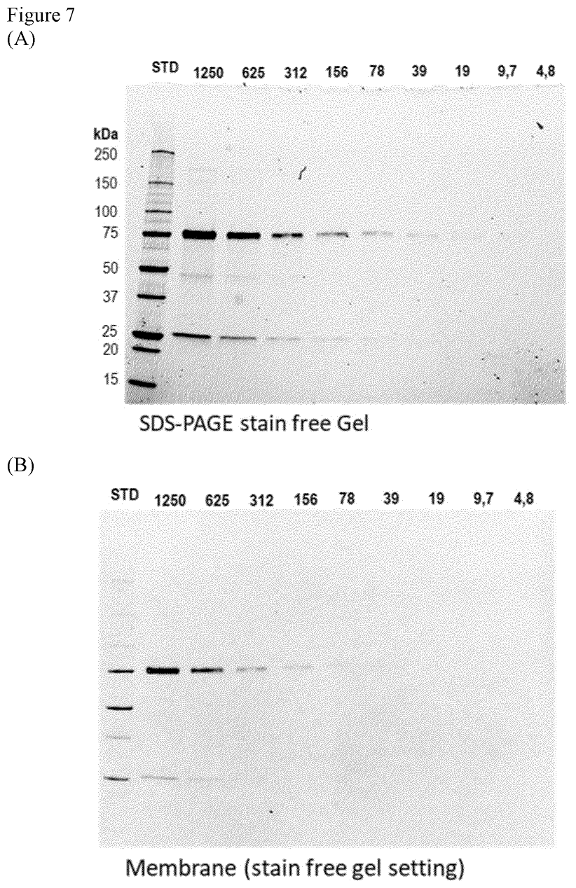

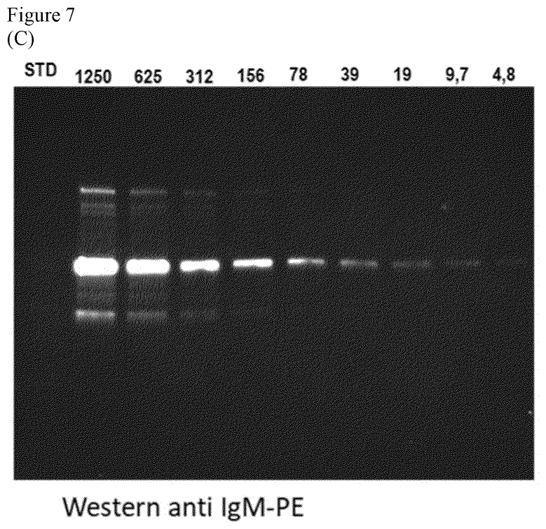

Purified human IgM was titrated (1250 to 4.8 ng/well) to see the detection limit of the assay. DTT reduced human IgM was separated on a stain-free SDS-PAGE gel. The reduced heavy chain is positioned at 75 kDa and visible down to 39 ng/well with the used settings. After transfer to nitrocellulose membrane the relocation of the protein was checked using the stain-free gel settings on the nitrocellulose membrane. The membrane was blocked and developed with PE-conjugated donkey anti-human IgM (One Lambda). The 75 kDa heavy chains are visible down to 19 ng/well. Some bands at the highest IgM concentration are also visible at approx. 200 kDa and 50 kDa (see FIG. 7).

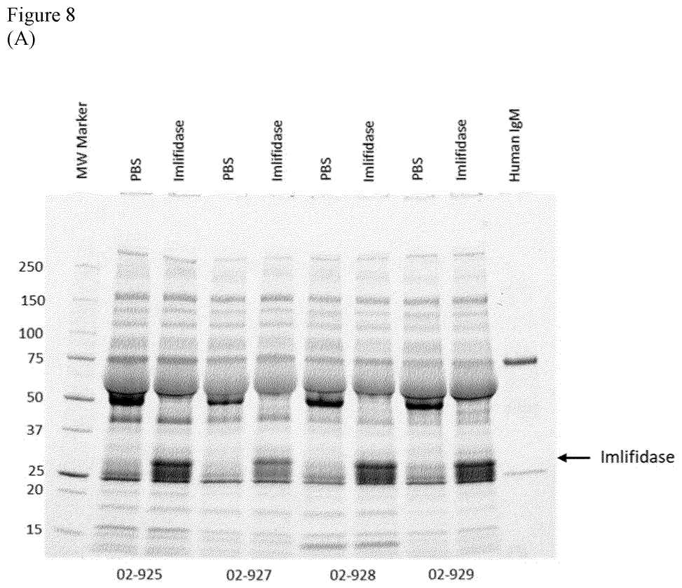

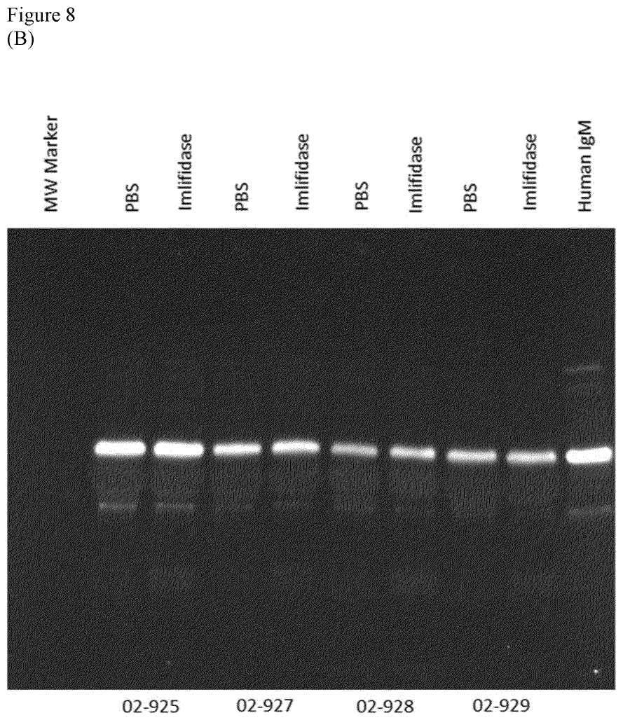

Sera from 4 sensitised ESRD patients (02-925, 02-927, 02-928 and 02-929) treated in vitro with imlifidase, respectively PBS. Serum samples and purified IgM were DTT treated and separated on an SDS-PAGE gel and transferred to nitrocellulose membrane, blocked and developed with PE-conjugated donkey anti-human IgM (One Lambda). The 75 kDa heavy chain band is clearly stained and no difference visible with or without imlifidase treatment (see FIG. 8).

Measurements of Rheumatoid Factor (RF)

All serum samples investigated for this report were diagnosed negative for rheumatoid factor (IgM-RF).

Measurements for Immunoglobin (Ig) Class and IgG Subclasses after Purification with CaptureSelect Matrix

Serum samples from two healthy volunteers were measured for immunoglobulin class and IgG subclasses pre-purification with CaptureSelect Matrix and post-purification in order to determine whether purification by CaptureSelect IgG-CH1 Affinity Matrix affects the composition of Ig in human serum. The results are shown in Table B:

| | | Human serum | | Human serum |
|---|---|---|---|---|
| Immunoglobulin | Human | A1 | Human | B1 |
| (% distribution) | serum A | (IgG depleted) | serum B | (IgG depleted) |
| IgG (75%) | 9.48 g/L | <0.33 g/L | 15.7 g/L | <0.33 g/L |
| (ref 6.7-14.5 g/L) | | | | |
| IgG1 (66%) | 4.32 g/L | <0.73 g/L | 9.09 g/L | <0.73 g/L |
| (ref 2.8-8.0 g/L) | | | | |
| IgG2 (23%) | 3.09 g/L | <0.10 g/L | 3.21 g/L | 0.10 g/L |
| (ref 1.15-5.70 g/L) | | | | |
| IgG3 (7%) | 0.67 g/L | <0.09 g/L | 1.37 g/L | <0.09 g/L |
| (ref 0.24-1.25 g/L) | | | | |
| IgG4 (4%) | 0.18 g/L | 0.07 g/L | <0.07 g/L | <0.07 g/L |
| (ref 0.05-1.25 g/L) | | | | |
| IgA (15%) | 2.51 g/L | 1.27 g/L | 1.88 g/L | 0.96 g/L |
| (ref 0.88-4.5 g/L) | | | | |
| IgM (10%) | 1.30 g/L | 0.66 g/L | 0.64 g/L | 0.30 g/L |
| (ref 0.27-2.1 g/L) | | | | |

### Conclusions

Anti-HLA IgM signals were reduced after treatment with an IgG cysteine protease in some patients. However, this HLA-IgM signal reduction was not observed in IgG-depleted sera. The initially high anti-HLA IgM signals are likely to be the result of IgG-complexed IgM on the surface of LABScreen HLA beads. Therefore, human IgM is not cleaved by the IgG cysteine protease, but IgG-complexed IgM can interfere with methods for detecting IgM and can produce false signals. The reduction in anti-HLA IgM signals observed in certain patients after treatment with an IgG cysteine protease shows that IgG cysteine proteases are able to cleave the problematic complexes.

The abstract by Zhang et al. (2019, Am J Transplant. 19 (suppl 3)) claims that the IgG cysteine protease imlifidase can reduce pathological anti-HLA IgM, in which case imlifidase would not be useful for detecting any antigen-specific IgM and would not be useful for treating diseases mediated by complexes of IgG and IgM. Strikingly, the present study demonstrates that IgG cysteine proteases are instead capable of reducing antigen complexes consisting of IgG and IgM by cleaving IgG. The data also highlight that methods for depleting IgG are necessary if one wishes to be sure to obtain a true IgM signal in assays for detecting IgM antibodies, and that that using an IgG degrading cysteine protease could be particularly suitable for this purpose. Similarly, IgG cysteine proteases are likely to be useful in treatments aimed at reducing antibody complexes consisting of IgG and other Ig isotypes e.g, IgM, IgA or IgE.

### Example 2

### Introduction

The aim of this study was to further assess the specificity of imlifidase by investigating if there is any effect on human anti-HLA IgM in sensitized patients with ESRD or if there could be any indirect effects of imlifidase on anti-HLA IgM SAB-signals in assays routinely used in the clinic. The data and samples obtained in Example 1 were analysed in more detail, and further experiments were performed.

### Material and Methods

Serum Samples from Sensitized Patients with End Stage Renal Disease (ESRD)

Patients with ESRD who are on dialysis and on the waiting list for a kidney transplant at the Department of Surgical Sciences, Section of Transplant Surgery at Uppsala University Hospital, Sweden, were eligible for the clinical trial (13-HMedIdeS-02; NCT02224820), if they had antibody reactivity against ≥2 identified anti-HLA antibodies of which one or more were above 3,000 mean fluorescence intensity (MFI) in a single antigen bead analysis on at least two separate occasions. Eight patients at enrolment received 1 or 2 intravenous infusions of imlifidase on consecutive days (0.12 mg/kg body weight×2 [n=3]; 0.25 mg/kg×1 [n=3], or 0.25 mg/kg×2 [n=2]). The clinical trial was conducted in accordance with the ethical principles that have their origins in the Declaration of Helsinki; all ethical and regulatory approvals were available before any patient was exposed to any study related procedure. IRB approval for use of clinical samples for science research was approved (EudraCT Number: 2013-005417-13; Diary Number: 2014/131, approved EPN 2014-04-16), and all samples were remarked and anonymized.

In Vitro Treatment with Imlifidase

Imlifidase (Idefirix®, Hansa Biopharma AB, Sweden) was used for in vitro treatments. The 35 kDa monomeric active substance imlifidase is an Escherichia coli expressed recombinant protein. For the reported in vitro studies imlifidase was diluted in PBS alone or PBS (BSA 0.05%) to a final concentration of 0.002-200 μg/mL and samples were allowed to incubate for either 1 or 2 hours at 37° C.

SDS-PAGE and Immunoblotting

Imlifidase treated purified human IgM (1 mg/mL) (#16-16-090713-M, Athens Research & Technology) or human IgG (1 mg/mL) (IVIg, Gamunex®) and IgM in serum samples from sensitized patients with ESRD were separated after DTT (#D9163, Sigma) treatment (only for IgM) on 4-20% Mini-PROTEAN TGX Stain-Free gels (#456-8093, Bio-Rad) in Tris-Glycine-SDS buffer (#161-0732, Bio-Rad). The gels were transferred to 0.45 μm nitrocellulose membranes (#LC2001, Novex) and blocked in 5% non-fat milk (Skim milk powder, OXOID) prior to incubation with PE-conjugated donkey anti-human IgM (#IGM-PEC1, One Lambda) in PBS-Tween. Membranes were washed and analysed in blot settings suitable for phycoerythrin (PE). Signals were acquired using a ChemiDoc MP system (Bio-Rad).

IgG Purification Using CaptureSelect™ Affinity Matrix

CaptureSelect™ IgG-CH1 Affinity Matrix (#194320005, ThermoFisher Scientific) was used for purification of serum samples. The CaptureSelect™ Affinity Matrix purifies recombinant human Fab fragments and IgG from complex source materials in a single step. The affinity matrix recognizes all four subclasses of IgG (IgG1, IgG2, IgG3 and IgG4), independent of the light chain type (kappa/lambda). Serum samples were analysed on SDS-PAGE for evaluation and purification of IgG.

Single Antigen Bead HLA Assay for Class I and II Anti-HLA Antibodies (One Lambda)

Serum samples from sensitized patients with ESRD treated with imlifidase at 0.12 or 0.25 mg/kg were investigated. Study samples, pre-dose and 24 hours post-dose imlifidase, were studied. All serum samples were pre-treated with ethylenediaminetetraacetic acid (Ultrapure 0.5 M EDTA, pH 8.0, REF 15575-038, Invitrogen, Grand Island, N.Y., USA) with a final solution by 5 mM solution to overcome the prozone effect. Sera were tested for both HLA class I and class II anti-HLA antibody using Single Antigen Beads on the Luminex platform (LABScreen Single Antigen, #LS1A04, lot 010, #LS2A01 lot 012, One Lambda, Canoga Park, Calif.). The serum was first incubated with LABScreen beads for 30 minutes, and the beads were then washed three times with wash buffer. Phycoerythrin (PE)-conjugated goat anti-human IgG (#LS-AB2, One Lambda, Canoga Park, Calif.) or PE-conjugated donkey anti-human IgM (#IGM-PEC1, One Lambda, Canoga Park, Calif.) was added and incubated for 30 minutes, washed two times. The LABScan 200 analyzer was used to detect the fluorescent emission of PE from each bead. The reaction pattern of the test serum was compared to the lot-specific worksheet defining the antigen array and assigned the HLA specificity. The results were interpreted by using HLA Fusion software (version 4.3, One Lambda) and expressed as raw data mean fluorescence intensity (MFI).

Data Analysis for Single Antigen Bead HLA Assay

Mean fluorescence intensity (MFI) values for each antibody specificity were determined using the baseline formula within HLA Fusion 4.3 software (OneLambda). A baseline MFI threshold of 3,000 was used to assign positive reactions with all reporter antibodies (IgG-PE and IgM-PE). Baseline raw MFI data for each test serum was transferred into a Microsoft Office Excel software (Microsoft) spreadsheet for analysis and comparison between the different test conditions.

Rheumatoid Factor (RF)

Serum samples from sensitized patients with ESRD were diagnosed for rheumatoid factor (IgM-RF) (Labmedicin Skåne, Clinical Immunology and Transfusion Medicine, Lund, Sweden).

Immunoglobin (Ig) Class and IgG Subclass Analysis

Serum samples from two healthy volunteers were measured for immunoglobulin class and IgG subclasses pre-purification with CaptureSelect™ Matrix and post-purification (Labmedicin Skåne, Clinical Immunology and Transfusion Medicine, Lund, Sweden).

### Results

Imlifidase Cleaves Circulating Anti-HLA IgG Antibodies

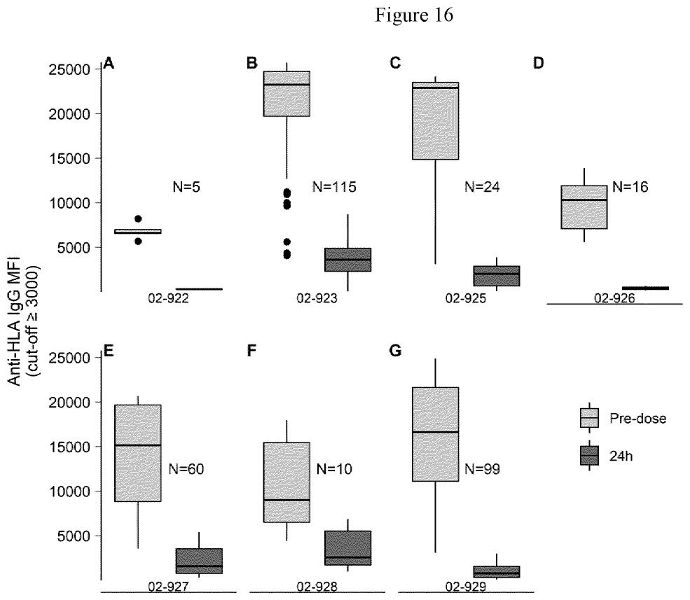

Pre-imlifidase and 24 hours post-imlifidase serum samples from sensitized patients with ESRD (N=7) were studied. The ability of imlifidase to cleave IgG including anti-HLA IgG antibodies was confirmed (FIG. 16), as established in previous studies (Jordan et al., 2017; Lonze et al., 2018; Lorant et al., 2018; Jordan et al., 2020, Schinstock, 2020). A clear reduction of the level of circulating anti-HLA IgG was observed in all the tested patients (FIG. 16).

Treatment with Imlifidase has Impact on the Assay Signal in the Anti-HLA IgM SAB Assay when Using Neat Serum

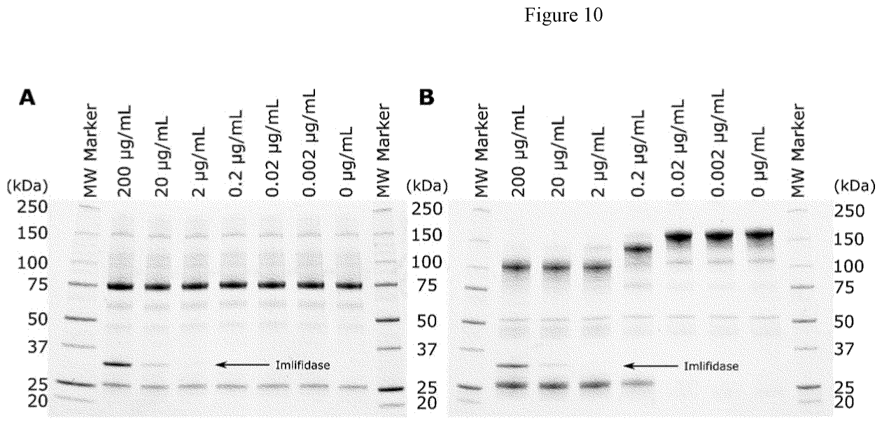

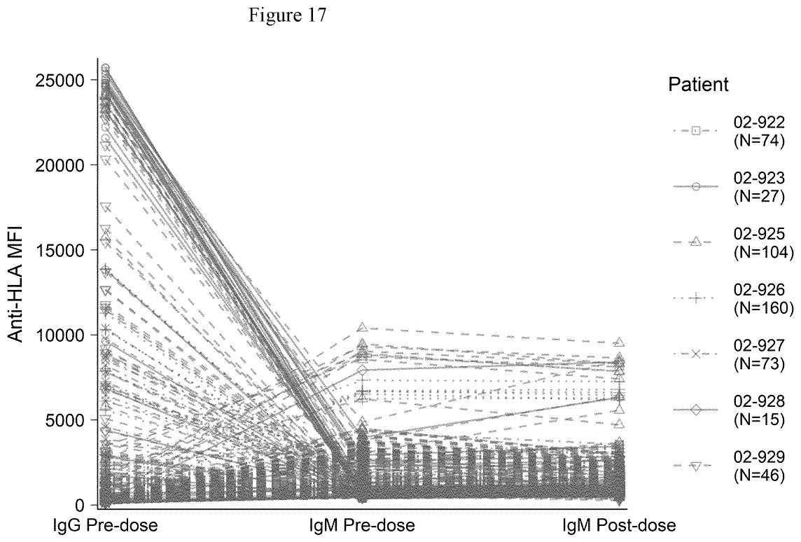

The samples were also analysed for SAB-HLA IgM (FIG. 10). In 2 out of 7 patients tested, a reduction in anti-HLA IgM signal between pre-dose and 24 hours after imlifidase treatment was observed (FIGS. 10E and 10G; patients 02-927 and 02-929). There was on average approximately 26 times (32 antigens) and 15 times (7 antigens) decrease in the antigen MFI values for patient 02-927 and 02-929, respectively. The common denominator for all these antigens was that the intensity of the corresponding pre-dose value for the beads was very high (minimum 11000 MFI) for the same antigens in the IgG SAB-HLA assay.
In patient 02-923 and 02-925 (FIGS. 10B and 10C) there were some beads that increased in the SAB-HLA IgM assay at 24 hours post dosing. For patient 02-923 the increase was observed in beads containing the HLA-A and HLA-B antigens (13 antigens) with an average increase of 5 times the pre-dose value. For patient 02-925 the increase was observed in beads containing the HLA-DQ antigen (10 antigens) with an average increase of 2 times the pre-dose value. The pre-dose value for the same antigens in the IgG SAB-HLA assay beads was extremely high (minimum 22000 MFI) which is in the range of the expected saturation of the beads (which generally occurs at >20000 MFI) (McCaughan et al., 2019). The presence of high levels of IgG might therefore negatively impact the number of available binding sites for the low affinity IgM in the assay. When the SAB-HLA bead was low in the IgG assay there was in general little difference in the IgM assay between the pre-dose and 24 hours samples (FIG. 17).

Purified Human IgM is Unaffected by Imlifidase Treatment

SDS-PAGE was used to test if imlifidase has the ability to cleave purified human IgM. No cleavage of purified human IgM by imlifidase was seen even at high concentrations up to 200 μg/mL (FIG. 10A). In contrast, human IgG with the same treatment was cleaved to scIgG already at 0.2 μg/mL and IgG was fully cleaved at 2 μg/mL (FIG. 10B).

No Cleavage of IgM in Human Serum Treated with Imlifidase

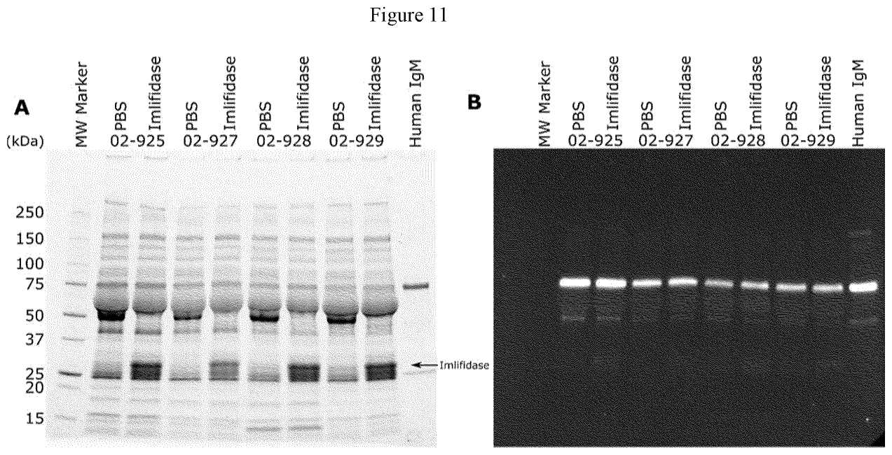

Cleavage of human IgM in serum samples from 4 sensitized ESRD patients (02-925, 02-927, 02-928 and 02-929) were investigated after in vitro treatment with high concentration of imlifidase. After imlifidase treatment serum samples (and purified IgM) were reduced, separated on SDS-PAGE (FIG. 11A) and subjected to Western blot using a PE-conjugated anti-human IgM (FIG. 11B). The intact 75 kDa heavy chain band of intact human IgM was clearly stained, and no intensity differences are visible with or without imlifidase treatment clearly indicating that IgM in serum is not cleaved by imlifidase in vitro.

Purification of IgG from Serum Using CaptureSelect™ Affinity Matrix

All serum samples from the seven patients were successfully depleted of IgG using a CaptureSelect™ affinity matrix. No IgG could be detected on the gels compared to untreated serum samples (FIG. 4). All serum samples investigated for this report were diagnosed negative for rheumatoid factor (IgM-RF) according to the clinical assay used (Analysportalen Skåne, Lund Sweden).

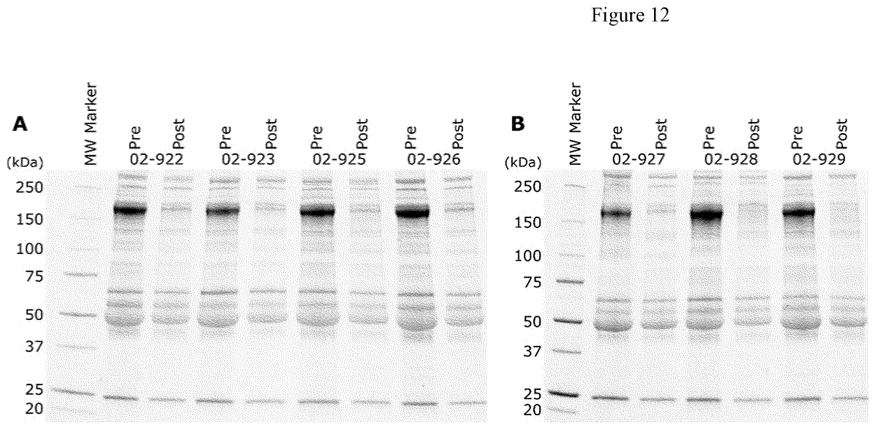

To determine that the CaptureSelect matrix mainly removes IgG and no other immunoglobulins, serum samples were measured for IgG, IgA and IgM and the IgG subclasses. Due to the amounts of serum required for this analysis healthy volunteers were used instead of the patient samples as a proof of principle in combination with the gels (FIG. 12). All subclasses of IgG were depleted below the detection limit. The other immunoglobulin classes were reduced approximately 50% of their initial value due to the procedure.

Treatment with Imlifidase has No Impact on Anti-HLA IgM Antibodies in IgG Depleted Serum

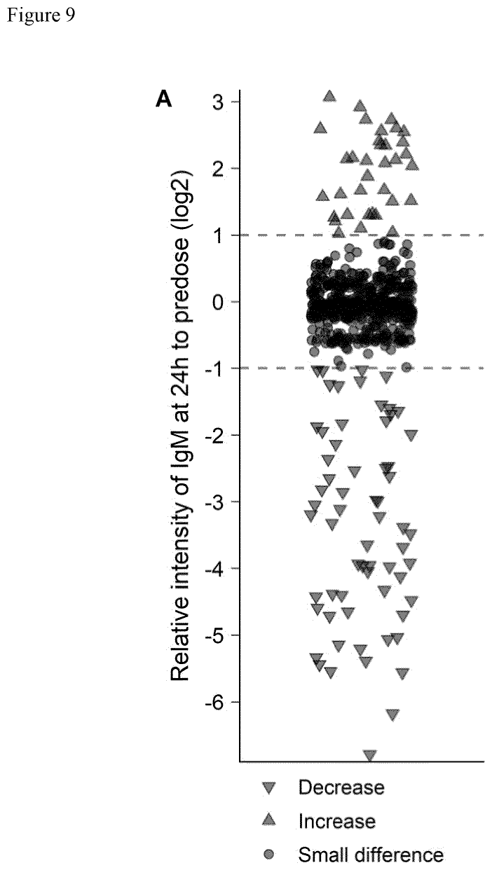

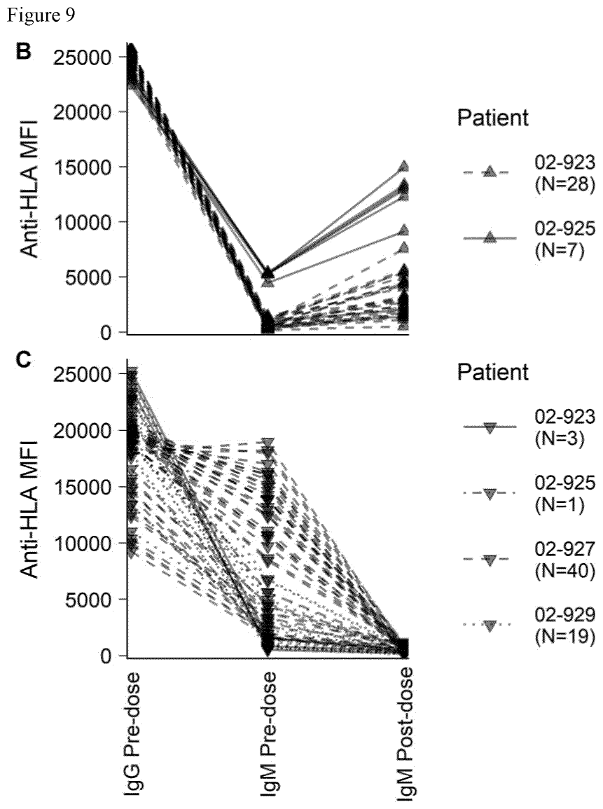

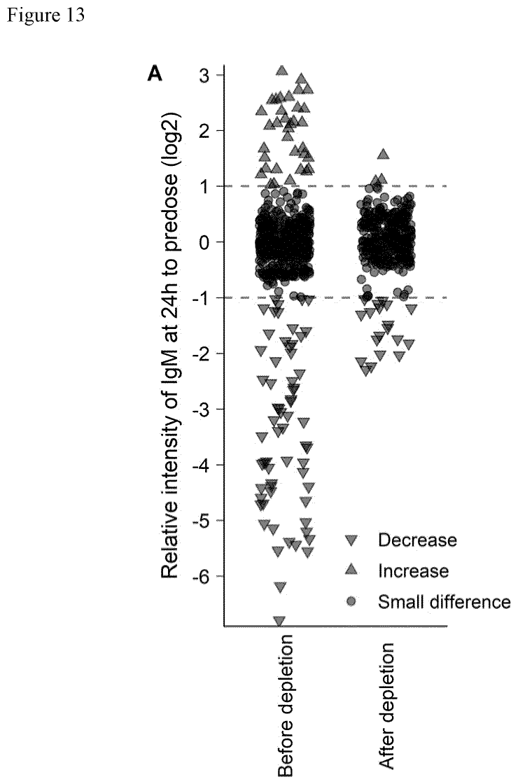

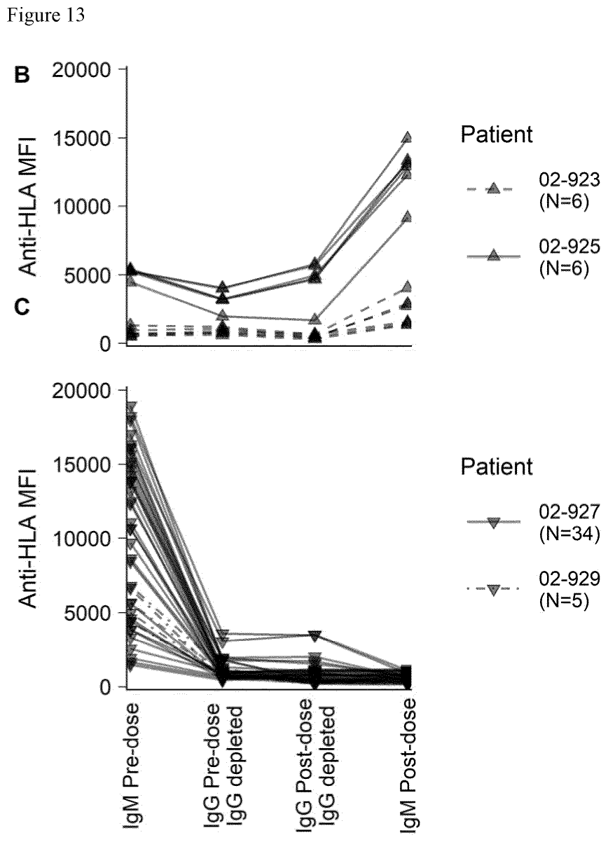

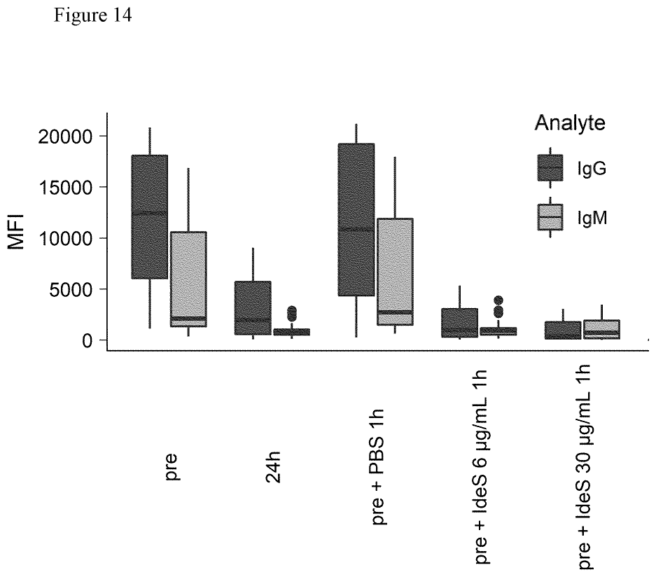

The effect between pre-dose and post-dose observed in FIG. 1 was completely removed in the IgG depleted samples (FIG. 13). The signal of anti-HLA IgM was in general lower (˜80%) for the IgG depleted samples in comparison with before depletion (compare FIG. 9 and FIG. 13), possibly due to the dilution from the washing in the depletion protocol. For patient 02-925 a similar pattern with increase in the 24 hours sample in comparison to the pre-dose was observed after depletion (FIG. 13) as before depletion (FIG. 9B) for some HLA-DQ antigens, but with reduced effect 46% increase instead of 115%. For patient 02-927 (FIG. 13) there were still 10 beads that were higher (3 times on average) in the IgG depleted pre-dose compared to the 24 hours sample. These were the beads that had the highest reduction before depletion (FIG. 9C), for these beads the mean MFI were 15186 and 1023 before and after IgG depletion, respectively. The 24 h sample MFI for the same beads was the same before and after IgG depletion. The same pattern was seen for 02-929, but with fewer beads. That the reduction of these beads still exist may be due to these IgM antibodies are co-depleted with the IgG due to their affinity to IgG and not to the HLA. In vitro treating a serum sample with imlifidase prior to SAB-HLA IgG/IgM testing generates results comparable to IgG depletion (FIG. 14).

### DISCUSSION

The data presented in Example 2 confirm the conclusions made in Example 1. In this study the specificity of Imlifidase activity with regards to IgM was assessed. We can confidently conclude that Imlifidase even in vitro in a PBS buffer system under ideal conditions has no detectable activity on IgM.

The data presented here demonstrate that the decrease in IgM signal following imlifidase treatment that occurs in the SAB-HLA IgM assay is likely due to an artefact which is resolved by the imlifidase treatment.

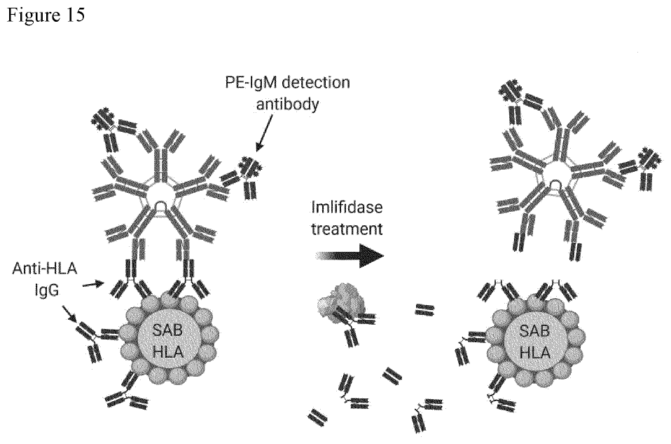

Our proposed mechanism is that IgG-DSA complexed IgM bind to the SAB-HLA beads (FIG. 15). The IgM detection antibody recognizes IgM in the complex, resulting in an artefactual IgM signal. After imlifidase treatment, these IgG-IgM complexes will be cleaved, and a much lower but true IgM signal will be recorded in the SAB HLA assay. These data therefore demonstrate the utility of imlifidase treatment when detecting IgM antibodies.

This model is supported by several of our observations;

Firstly, there is no detectable enzymatic activity on human IgM under ideal conditions even with very high concentrations of imlifidase;

Secondly, the appearance of the artefact in some patients can be clearly linked to IgM and the presence of especially high IgG DSA levels. This combination appears to be a prerequisite for the generation of the false positive IgM signals and it also disappeared after treating a pre-dose patient serum sample with imlifidase in vitro prior to running the test.

Thirdly, the false positive signal in the pre-dose samples also vanishes in all the observed cases after IgG depletion. Furthermore, the reduction of this false positive IgM signal can primarily be seen on antigen beads towards which there is a particularly high level of IgG DSA signal in the SAB-IgG assay.

It is interesting that the presence of RF could not be detected in the patients when using a standard clinical assay.

It is clear from our results is that the enzymatic activity of imlifidase efficiently can dissolve IgM and IgG complexes. This could have clinical implications in patients where IgG and IgM complex formation is contributing to exacerbation of the symptoms of an autoimmune disease.

Interestingly and in contrast to the false positive signal just described, in some patients, especially when looking at antigen beads having a high IgG signal, we could also observe an increase in the IgM signal post imlifidase treatment. This is likely explained by a reduced steric hindrance of DSA IgG that after the decrease in serum Ig-levels following imlifidase treatment enables the DSA IgM to now bind and generate a signal.

At high IgG DSA concentration there therefore appears to be a risk for the generation both of false positive as well as false negative IgM DSA signals in the SAB-HLA IgM test. This could in the worst-case scenario jeopardize a physicians ability to take the right clinical decision or lead to a decision based on inaccurate data.

The IgM assay is currently used sparingly in clinical decision-making but the presence of IgG IgM aggregates in the IgG SAB HLA could in a similar manner potentially falsely increase IgG DSA signals and the results could lead to an unnecessary long time on the waiting list for these patients.

Some HLA centres laboratories employ methods such as the addition of EDTA, heat inactivation, addition of DTT (Dithiothreitol) and serial dilutions to overcome problems with the prozone effect and these methods can improve the predictive accuracy of the SAB-HLA assay. Our results indicate that in vitro treatment of test samples with imlifidase or IgG-depletion prior to running the SAB-HLA test can improve the signal on some of the beads and decrease the risk of false negative results and at the same time reduce the risk of a false positive IgM signal. In vitro treating samples with imlifidase prior to testing could therefore be a potential new method for improving the accuracy and reliability of the IgM SAB-HLA assay. To conclude the effect we have observed of imlifidase on IgG/IgM complexes is expected to have both clinical and diagnostic uses.

1. A method for detecting IgM antibodies, IgA antibodies or IgE antibodies in a sample comprising:
a. contacting said sample with an IgG cysteine protease under conditions which permit IgG cysteine protease activity to occur; and
b. contacting said sample with an agent suitable for detecting IgM antibodies, IgA antibodies or IgE antibodies.

2. The method of claim 1, wherein said IgG cysteine protease is an IdeS or IdeZ polypeptide.

3. The method of claim 1, wherein said IgG cysteine protease is a polypeptide having a sequence that is at least 80% identical to SEQ ID NO: 2, 4 or 5, such as at least 85%, 90%, 95% or 99% identical, or wherein said IgG cysteine protease comprises or consists of the sequence of any one of SEQ ID NOs: 6 to 25 and 55 to 69, optionally wherein said sequence includes an additional methionine at the N terminus and/or a histidine tag at the C terminus.

4. The method of claim 1, wherein said agent suitable for detecting IgM antibodies, IgA antibodies or IgE antibodies is an anti-IgM antibody, an anti-IgA antibody or an anti-IgE antibody, optionally fixed to a solid substrate, such as a bead or a plate.

5. The method of claim 1, wherein said sample is contacted with an antigen of interest, such as a Human Leukocyte Antigen (HLA), an erythrocyte antigen or a drug antigen, under conditions that permit isolation of antibodies that bind specifically to the antigen of interest, optionally wherein said antigen of interest is fixed to a solid substrate, such as a bead or a plate.

6. The method of claim 1, wherein said sample is a sample obtained from a patient, such as a serum sample, optionally wherein said sample is obtained from a patient that has been diagnosed as requiring an organ transplant, or a patient diagnosed with a disease selected from Table A.

7. The method of claim 1, wherein the method is an ELISA, a single antigen bead assay, a surface plasmon resonance assay, a nephelometry assay or a bio-layer interferometry assay.

8. A kit for practising the method of claim 1, comprising an IgG cysteine protease and an agent for detecting an IgM antibody, an IgA antibody or an IgE antibody, optionally additionally comprising an antigen of interest, such as a Human Leukocyte Antigen (HLA), an erythrocyte antigen or a drug antigen.

9. A method for treating a disease or condition in a subject, comprising administering an IgG cysteine protease:
(a) wherein the disease or condition is mediated in whole or in part by pathogenic IgM antibodies, pathogenic IgA or pathogenic IgE antibodies; or
(b) wherein the disease or condition is mediated in whole or in part by pathogenic IgG antibodies, and wherein the subject is determined to exhibit anti-IgG IgM antibodies, anti-IgG IgA antibodies or anti-IgG IgE antibodies; or
(c) wherein the disease or condition is mediated in whole or in part by complexes of IgG antibodies and IgM, IgA or IgE antibodies.

10. (canceled)

11. (canceled)

12. The method of claim 9, wherein the pathogenic IgM antibodies, pathogenic IgA, pathogenic IgE antibodies or pathogenic IgG antibodies are anti-HLA antibodies, anti-erythrocyte antigen antibodies or anti-drug antibodies.

13. The method of claim 9, wherein said subject does not exhibit significant levels of anti-self IgG antibodies, or wherein said subject is determined to exhibit elevated levels of pathogenic IgM antibodies, pathogenic IgA antibodies or pathogenic IgE antibodies.

14. The method of claim 9, wherein said disease or condition is selected from the list consisting of Table A.

15. The method of claim 9, wherein the disease or condition mediated in whole or in part by complexes of IgG antibodies and IgM, IgA or IgE antibodies is selected from the list consisting of vasculitis, granulomatosis with polyangiitis (Wegener's granulomatosis), IgA nephropathy and systemic lupus erythematosus.
